SEQUENCE LISTING (43 sequences)

SEQ ID NO: 1 - DNA, 27 bp, Artificial sequence
  Synthetic primers
aaaaacgaacggctgatgaccaaactc

SEQ ID NO: 2 - DNA, 25 bp, Artificial sequence
  Synthetic primers
aaaaaggaggtggccatcgtggaag

SEQ ID NO: 3 - DNA, 30 bp, Artificial sequence
  Synthetic primers
aaaaacagtgtgttgattgtggaggacgag

SEQ ID NO: 4 - DNA, 28 bp, Artificial sequence
  Synthetic primers
aaaaagtggaatcaaagccctccttgcg

SEQ ID NO: 5 - DNA, 34 bp, Artificial sequence
  Synthetic primers
aaaaacaaatacttaatcgagcaatatggcggcg

SEQ ID NO: 6 - DNA, 34 bp, Artificial sequence
  Synthetic primers
aaaaagcttatacaccatcgtcgcaatcatctcc

SEQ ID NO: 7 - DNA, 22 bp, Artificial sequence
  Synthetic primers
cgaacggctgatgaccaaactc

SEQ ID NO: 8 - DNA, 20 bp, Artificial sequence
  Synthetic primers
ggaggtggccatcgtggaag

SEQ ID NO: 9 - DNA, 25 bp, Artificial sequence
  Synthetic primers
cagtgtgttgattgtggaggacgag

SEQ ID NO: 10 - DNA, 23 bp, Artificial sequence
  Synthetic primers
gtggaatcaaagccctccttgcg

SEQ ID NO: 11 - DNA, 29 bp, Artificial sequence
  Synthetic primers
caaatacttaatcgagcaatatggcggcg

SEQ ID NO: 12 - DNA, 29 bp, Artificial sequence
  Synthetic primers
gcttatacaccatcgtcgcaatcatctcc

SEQ ID NO: 13 - DNA, 22 bp, Artificial sequence
  Synthetic primers
ggcaggttggtatcaaggttac

SEQ ID NO: 14 - DNA, 22 bp, Artificial sequence
  Synthetic primers
cctaagggtgggaaaatagacc

SEQ ID NO: 15 - DNA, 20 bp, Artificial sequence
  Synthetic primers
actgggcatgtggagacaga

SEQ ID NO: 16 - DNA, 26 bp, Artificial sequence
  Synthetic primers
cgatgtgtactgagatcccctatccg

SEQ ID NO: 17 - DNA, 19 bp, Artificial sequence
  Synthetic primers
ggcctttgtcaccgacgcc

SEQ ID NO: 18 - DNA, 26 bp, Artificial sequence
  Synthetic primers
aacgtctttcaggtcgagtacgcctt

SEQ ID NO: 19 - DNA, 21 bp, Artificial sequence
  Synthetic primers
cagagcgtagcgatgaggtgg

SEQ ID NO: 20 - DNA, 20 bp, Artificial sequence
  Synthetic primers
gcctcaaagccctccttgcg

SEQ ID NO: 21 - DNA, 20 bp, Artificial sequence
  Synthetic primers
gaggacgaggagtcgctggc

SEQ ID NO: 22 - DNA, 21 bp, Artificial sequence
  Synthetic primers
ggcggctcttcgttacttaaa

SEQ ID NO: 23 - DNA, 22 bp, Artificial sequence
  Synthetic primers
gaactcctcggtccctatatca

SEQ ID NO: 24 - DNA, 24 bp, Artificial sequence
  Synthetic primers
tacctgacaaagtgatcgggctgc

SEQ ID NO: 25 - DNA, 20 bp, Artificial sequence
  Synthetic primers
actgggcatgtggagacaga

SEQ ID NO: 26 - DNA, 26 bp, Artificial sequence
  Synthetic primers
aacgtctttcaggtcgagtacgcctt

SEQ ID NO: 27 - DNA, 20 bp, Artificial sequence
  Synthetic primers
gaggacgaggagtcgctggc

SEQ ID NO: 28 - DNA, 24 bp, Artificial sequence
  Synthetic primers
tacctgacaaagtgatcgggctgc

SEQ ID NO: 29 - DNA, 22 bp, Artificial sequence
  Synthetic primers
cagagcgtagcgatgaggtggg

SEQ ID NO: 30 - DNA, 27 bp, Artificial sequence
  Synthetic primers
aaaaacagagcgtagcgatgaggtggg

SEQ ID NO: 31 - DNA, 29 bp, Artificial sequence
  Synthetic primers
gcttatacaccatcgtcgcaatcatctcc

SEQ ID NO: 32 - DNA, 18 bp, Artificial sequence
  Synthetic primers
ctcaaagccctccttgcg

SEQ ID NO: 33 - DNA, 23 bp, Artificial sequence
  Synthetic primers
cgatgtgtactgagatcccctat

SEQ ID NO: 34 - DNA, 16 bp, Artificial sequence
  Synthetic primers
ctttgtcaccgacgcc

SEQ ID NO: 35 - DNA, 26 bp, Artificial sequence
  Synthetic primers
aacgtctttcaggtcgagtacgcctt

SEQ ID NO: 36 - DNA, 18 bp, Artificial sequence
  Synthetic primers
agagcgtagcgatgaggt

SEQ ID NO: 37 - DNA, 18 bp, Artificial sequence
  Synthetic primers
ctcaaagccctccttgcg

SEQ ID NO: 38 - DNA, 21 bp, Artificial sequence
  Synthetic primers
ggcggctcttcgttacttaaa

SEQ ID NO: 39 - DNA, 32 bp, Artificial sequence
  Synthetic primers
tttttgaattctcaatcagccattggtaggtc

SEQ ID NO: 40 - DNA, 34 bp, Artificial sequence
  Synthetic primers
tttttgaattcagctgcaatatcctgtaaaggtc

SEQ ID NO: 41 - DNA, 20 bp, Artificial sequence
  Synthetic primers
tgaccagtgtgttgattgtg

SEQ ID NO: 42 - DNA, 18 bp, Artificial sequence
  Synthetic primers
gagagctgccggaccatc

SEQ ID NO: 43 - DNA, 19 bp, Artificial sequence
  Synthetic primers
aggacgaggagtcgctggc

(19) United States
(12) Patent Application Publication
(10) Pub. No.: US 2018/0016623 A1
(43) Pub. Date: Jan. 18, 2018

(54) COMPOSITIONS AND METHODS FOR DETECTING NUCLEIC ACIDS IN SPUTUM

(71) Applicants: Northwestern University, Evanston, IL (US); University of Cape Town, Cape Town (ZA)

(72) Inventors: Sally M. McFall, Evanston, IL (US); Jennifer L. Reed, Evanston, IL (US); Mark P. Nicol, Cape Town (ZA); Zachary Walker, Aurora, CO (US); Matthew A. Butzler, Elk Grove Village, IL (US)

(21) Appl. No.: 15/650,517

(22) Filed: Jul. 14, 2017

Related U.S. Application Data
(60) Provisional application No. 62/363,069, filed on Jul. 15, 2016.

(51) Int. Cl. C12Q 1/68; C12N 15/10

(52) U.S. Cl. CPC C12Q 1/6806; C12Q 1/689; C12N 15/1006; C12Q 2600/16; C12Q 2600/158

(57) ABSTRACT

This disclosure relates generally to methods and kits useful for preparing samples, extracting nucleic acids from samples (e.g., biological samples), and/or detecting nucleic acids (e.g., pathogen nucleic acids) in samples (e.g., samples obtained from a subject). In particular, compositions, kits, and methods are provided comprising detergents and proteinases to treat biological samples prior to extraction of nucleic acids. Also described is use of cations for improved efficiency of nucleic acid hybridization. The prepared nucleic acid is suitable for PCR assays including those described for detection of Mycobacterium tuberculosis.

### CROSS-REFERENCE TO RELATED APPLICATIONS

The present application claims priority to U.S. Provisional Patent Application Ser. No. 62/363,069 filed Jul. 15, 2016, which is hereby incorporated by reference in its entirety.

### FIELD

This disclosure relates generally to methods and kits useful for preparing samples, extracting nucleic acids from samples (e.g., biological samples), and/or detecting nucleic acids (e.g., pathogen nucleic acids) in samples (e.g., samples obtained from a subject). In particular, compositions, kits, and methods are provided comprising detergents and proteinases to treat biological samples prior to extraction of nucleic acids. Also described is use of cations for improved efficiency of nucleic acid hybridization. The prepared nucleic acid is suitable for PCR assays including those described for detection of Mycobacterium tuberculosis.

### BACKGROUND

Tuberculosis (TB), caused by Mycobacterium tuberculosis complex (MTBC) species, remains one of the deadliest infectious diseases with an estimated 9.6 million people falling ill, and 1.5 million people dying from TB globally in 2014 (WHO, Global Tuberculosis Report 2015, W. H. Organization, Editor 2015, World Health Organization: Geneva, Switzerland; incorporated by reference in its entirety). HIV-1 positive populations are particularly susceptible to TB and are 29 times more likely to die from active TB disease than HIV-1 negative populations. HIV-TB coinfection contributes substantially to TB-associated deaths worldwide, as 1.2 million (12%) individuals who developed TB were HIV positive, and 0.4 million co-infected patients died in 2014, accounting for 27% of the estimated 1.5 million deaths from TB (WHO, Global Tuberculosis Report 2015, W. H. Organization, Editor 2015, World Health Organization: Geneva, Switzerland). Accurate and timely diagnosis is the first step in providing care to patients and to prevent transmission.

Mycobacterial culture has long been the conventional gold standard test for TB diagnosis in high resource settings. It has high sensitivity (limit of detection ˜10 cfu/ml) (van Zyl-Smit et al., 2011, PLoS ONE, 6:e28815; incorporated by reference in its entirety), but the time-to-result is lengthy (ranging from 2-8 weeks) (The American Thoracic Society, 2000, Am J Respir Crit Care Med, 161:1376-1395; incorporated by reference in its entirety) and the sample preparation is technically challenging, prone to contamination and requires a BSL-3 laboratory facility. In most low resource settings, bacterial culture is unavailable, leaving sputum smear microscopy as the only direct bacteriological test available (Wejse, 2014, Lancet, 383:388-390; incorporated by reference in its entirety). The limit of detection (LOD) of the unconcentrated smear test is approximately 10,000 cfu/ml, and it has poor specificity especially in settings where non-tuberculosis mycobacteria are commonly isolated (The American Thoracic Society, 2000, Am J Respir Crit Care Med, 161:1376-1395; incorporated by reference in its entirety). Smear microscopy's poor clinical performance is particularly troublesome in settings with high HIV-1 incidence. The HIV-1 epidemic has led to a disproportionate increase in the reported rate of smear-negative TB patients, and co-infection with HIV-1 changes the presentation of smear-negative TB to more rapidly progressive disease with a high mortality rate (Colebunders and Bastian, 2000, Int J Tuberc Lung Dis, 4:97-107; Getahun et al., 2007, Lancet, 369:2042-2049; incorporated by reference in their entireties).

Nucleic acid amplification tests (NAT) have the potential to provide highly sensitive detection of paucibacillary forms of tuberculosis with rapid turn-around-time; however, the commercially available NATs are not yet meeting this need. The clinical sensitivity with smear-positive specimens is 98% when compared to culture; however, clinical sensitivity with smear-negative specimens is only 67% (Steingart et al., 2014, Cochrane Database Syst Rev, 1:CD009593; incorporated by reference in its entirety). This sensitivity can be improved to greater than 90% by testing the same patient 3 times (Helb et al., 2010, J Clin Microbiol, 48:229-237; incorporated by reference in its entirety). Suboptimal sensitivity is likely to lead to reduced test impact, as clinicians will continue to use empiric treatment in test-negative patients (Theron et al., 2014, Lancet, 383:424-435; incorporated by reference in its entirety) or alternatively may miss the diagnosis of TB in patients with paucibacillary TB, such as children and people living with HIV.

The performance of MTB NATs is greatly impacted by the lysis and DNA extraction methods utilized (Aldous et al., 2005, J Clin Microbiol, 43:2471-2473; Boddinghaus, et al., 2001, J Clin Microbiol, 39:3750-3752; incorporated by reference in their entireties). Leung, et al. (Leung et al., 2011, J Clin Microbiol, 49:2509-2515; incorporated by reference in its entirety) reported up to a 12.5-fold difference in DNA yield simply due to different cell lysis and DNA extraction protocols. Additionally, biological factors in sputum can cause PCR inhibition with a reported 5-fold decrease in analytical sensitivity of an MTB-specific PCR assay when comparing extraction from buffer to sputum (Shawar et al., 1993, J Clin Microbiol, 3161-65; incorporated by reference in its entirety). MTB NAT sample preparation can be performed by concentrating and purifying intact MTB from sputum, or by directly lysing MTB bacteria in sputum, followed by extraction of DNA. MTB in liquefied sputum can be concentrated through centrifugation followed by resuspension of the pellet in buffer, or MTB can be purified through filtration and washing (Boehme et al., 2010, New Eng J Med, 363:1005-1015; incorporated by reference in its entirety). However, logistical obstacles often thwart successful execution of these established sample processing methods. The sample volume of sputum is generally limited because of the high concentration of PCR inhibitors and nucleases found in the specimen can cause assay failure. Sputum itself is a viscous, heterogeneous mixture that contains high levels of nucleic acid, and concentrating by filtration limits the test volume because the filter will readily clog. Tests that rely on centrifugation to concentrate the bacilli from the liquefied sputum also neglect the free MTB DNA present in the supernatant (Pathak et al, 2007, BMC Microbiol, 7:83; incorporated by reference in its entirety), which may reduce the overall sensitivity of the assay.

What is needed are compositions, kits, and methods for performing pathogen-screening assays with improved efficiency of preparation and sensitivity of nucleic acid detection.

### SUMMARY

This disclosure relates generally to methods and kits useful for preparing samples, extracting nucleic acids from samples (e.g., biological samples), and/or detecting nucleic acids (e.g., pathogen nucleic acids) in samples (e.g., samples obtained from a subject). In particular, compositions, kits, and methods are provided comprising detergents and proteinases to treat biological samples prior to extraction of nucleic acids. Also described is use of cations for improved efficiency of nucleic acid hybridization. The prepared nucleic acid is suitable for PCR assays including those described for detection of Mycobacterium tuberculosis.

In some embodiments, a method for preparing a nucleic acid from a biological sample is provided. In some embodiments, the biological sample is from a subject suspected of being infected with a pathogen. In some embodiments, the biological sample is undiluted or diluted by addition of volume (e.g., comprising or consisting of lysis reagents (e.g., detergent, PK), buffer, etc.) amounting to less than 50% of the volume of the sample (e.g., <50%, <40%, <30%, <25%, <20%, <15%, <10%, <9%, <8%, <7%, <6%, <5%, <4%, <3%, <2%, <1%, etc.). In some embodiments, the sample comprises whole cells (e.g., whole cells from the subject, whole cells from a pathogen (e.g., bacterial pathogen, etc.), etc.). In some embodiments, the sample has not been subjected to one or more of sonication, ultrasonication, traditional lysis reagents, chaotropes, and/or other traditional lysis reagents. In some embodiments, the sample is selected from any biological sample, for example, blood (e.g., whole blood), sputum, tears, mucus, nasal washes, nasal aspirate, breath, urine, semen, saliva, peritoneal washings, ascites, cystic fluid, meningeal fluid, amniotic fluid, glandular fluid, lymph fluid, nipple aspirate, bronchial aspirate, bronchial brushing, synovial fluid, joint aspirate, organ secretions, cells, a cellular extract, and cerebrospinal fluid.

In some embodiments, the sample may contain one or more target sequences suitable for detecting the presence of a pathogen in the sample. In certain embodiments, the nucleic acid may contain one or more target sequences suitable for detecting the presence of a pathogen in the sample.

In some embodiments, provided herein are methods for preparing a biological sample for analysis, comprising: mixing the biological sample with thinning reagents comprising a detergent and a proteinase to produce a lysis mixture. In some embodiments, the detergent comprises is a neutral detergent and/or an anionic detergent. In some embodiments, the detergent comprises one or more anionic detergents selected from the group consisting of sodium dodecyl sulfate (SDS), N-lauroylsarcosine sodium salt, and sodium deoxycholate. In some embodiments, the detergent comprises one or more neutral detergents selected from the group consisting of CHAPS and CHAPSO. In some embodiments, the proteinase is proteinase K (PK). In some embodiments, the biological sample is selected from the group consisting of sputum, whole blood, mucus, nasal fluid, semen, saliva, amniotic fluid, and bronchial fluid. In some embodiments, the biological sample is sputum, the proteinase is PK, and the detergent comprises SDS. In some embodiments, the biological sample is undiluted prior to the mixing. In some embodiments, the thinning reagents are liquid reagents and have a volume that is less than 25% of the volume of the biological sample. In some embodiments, the thinning reagents are dried reagents. In some embodiments, the dried reagents are adhered to a surface of a vessel to which the biological sample is added. In some embodiments, cells within the biological sample have not been lysed prior to the mixing. In some embodiments, the biological sample has not been subjected to sonication or chaotropic agents. In some embodiments, methods further comprise heating the lysis mixture. In some embodiments, the lysis mixture is heated to 70° C. or greater. In some embodiments, the lysis mixture is heated to 90° C. or greater. In some embodiments, a bacterial pathogen is present in the biological sample. In some embodiments, the bacterial pathogen is selected from the list consisting of plasmodium, Mycobacterium tuberculosis, Salmonella typhi, Borrelia, Neisseria meningitides, and other bacterial or non-bacterial pathogens. In some embodiments, a bacterial pathogen present in the biological sample is not pathogenic in the lysis mixture.

In some embodiments, provided herein are methods comprising: (a) preparing a biological sample for analysis by the methods above and/or otherwise described herein; and (b) extracting nucleic acids from the lysis mixture. In some embodiments, extracting nucleic acids from the lysis mixture comprises: (i) combining the lysis mixture with hybridization buffer and at least one capture oligonucleotide to generate a capture solution, wherein the at least one oligonucleotide is specific for a target sequence and is linked to a capture moiety; and (ii) contacting the capture solution with a capture agent, wherein the capture agent binds to the capture moiety, wherein a capture complex is formed comprising (A) a nucleic acid comprising the target sequence, (B) the capture oligonucleotide, and (C) the capture agent. In some embodiments, the hybridization buffer and capture oligonucleotide are liquid reagents and are added to the lysis mixture. In some embodiments, the hybridization buffer and capture oligonucleotide are dried reagents. In some embodiments, the dried reagents are adhered to a surface of a vessel to which the lysis mixture is added. In some embodiments, the capture agent and the capture moiety form a stable non-covalent interaction upon contact. In some embodiments, the capture agent is bound to a solid surface. In some embodiments, the solid surface is a particle. In some embodiments, the particle is a magnetic particle (e.g., a paramagnetic particle). In other embodiments, extracting nucleic acids from the lysis mixture comprises: (i) combining the lysis mixture with hybridization buffer and at least one capture oligonucleotide to generate a capture solution, wherein the at least one oligonucleotide is specific for a target sequence and is bound to a solid surface (e.g., via a capture moiety on the oligonucleotide and a capture agent on the surface), wherein a capture complex is formed comprising (A) a nucleic acid comprising the target sequence and (B) the capture oligonucleotide attached to the solid surface. In some embodiments, the solid surface is a particle (e.g., magnetic particle, paramagnetic particle, etc.).

In some embodiments, methods further comprise: (iii) separating the capture complex from the capture solution. In some embodiments, separating the capture complex from the capture solution comprises washing the capture solution away from the capture complex. In some embodiments, separating the capture complex from the capture solution comprises withdrawing the capture complex from the capture solution (e.g., using a magnet to pull the magnetic particles from the capture solution, using a magnet to hold the magnetic particles static while the capture solution is removed, etc.). In some embodiments, methods further comprise: (iv) eluting the nucleic acid comprising the target sequence from capture complex into an elution buffer.

In some embodiments, provided herein are methods comprising: preparing a biological sample for analysis by the methods above and/or otherwise described herein; separating target nucleic acids from the biological sample by the methods above and/or otherwise described herein; and amplifying and/or analyzing the target nucleic acids by an assay described herein or otherwise known in the field. In some embodiments, the presence, absence, or level of a pathogen in the biological sample is determined.

In some embodiments, the method comprises mixing a biological sample with a thinning solution comprising a detergent and a proteinase to form a lysis mixture; adding at least a portion of the lysis mixture to a capture tube containing a hybridization buffer and at least one capture oligonucleotide to generate a hybridization sample solution, wherein the at least one oligonucleotide is specific for a target sequence and is linked to a capture moiety; adding a plurality of solid particles to the capture tube wherein the solid particles which bind to the capture moiety and incubating to form a particle complex with capture oligonucleotide, wherein the capture oligonucleotide hybridizes to the nucleic acid; washing the particle complex in conditions where the capture oligonucleotide remains hybridized to the nucleic acid; and eluting the nucleic acid from the particle complex into an elution buffer.

In some embodiments, the detergent is an anionic detergent. In certain embodiments, the detergent is a neutral detergent. In certain embodiments, the detergent is not a cationic detergent. In some embodiments, the anionic detergent is sodium dodecyl sulfate (SDS), N-lauroylsarcosine sodium salt, or sodium deoxycholate. In certain embodiments, the neutral detergent is CHAPS or CHAPSO. In certain embodiments, the proteinase is proteinase K (PK). In some embodiments, the lysis mixture is incubated at a first temperature ranging from about 40° C. to 75° C. (e.g., 40° C., 45° C., 50° C., 55° C., 60° C., 65° C., 70° C., 75° C., or ranges therebetween (e.g., 40° C. to 60° C., 45° C. to 55° C., 50° C. to 70° C., 50° C. to 60° C., etc.)) for a first time period. In certain embodiments, the first time period is about 5 to 40 minutes (e.g., 5 minutes, 10 minutes, 15 minutes, 20 minutes, 25 minutes, 30 minutes, 35 minutes, 40 minutes, or ranges therebetween (e.g., 5 to 15 minutes, 10 to 20 minutes, 10 to 15 minutes, 5 to 30 minutes, 5 to 10 minutes, etc.)).

In some embodiments, the lysis mixture is heated during the first time period with mixing. In some embodiments, the mixing is at 500 to 2000 rpm (e.g., 500 rpm, 600 rpm, 700 rpm, 800 rpm, 900 rpm, 1000 rpm, 1100 rpm, 1200 rpm, 1300 rpm, 1400 rpm, 1500 rpm, 1600 rpm, 1700 rpm, 1800 rpm, 1900 rpm, 2000 rpm, or ranges therebetween (e.g., 800 to 1500 rpm, etc.)).

In some embodiments, after incubating the lysis mixture at the first temperature for the first time period, the method further comprises incubating the lysis mixture at a second temperature of about 70° C. to 98° C. (e.g., 70° C., 72° C., 74° C., 76° C., 78° C., 80° C., 85° C., 90° C., 92° C., 94° C., 96° C., 98° C., or ranges therebetween (e.g., 70° C. to 94° C., 70° C. to 90° C., 80° C. to 98° C., 80° C. to 90° C., 85° C. to 98° C., etc.)) for a second time period. In certain embodiments, the incubating for the second time period is for a time of about of about 5 to 30 minutes (e.g., 5 minutes, 10 minutes, 15 minutes, 20 minutes 25 minutes, 30 minutes, or ranges therebetween (e.g., 5 to 15 minutes, 10 to 20 minutes, 10 to 15 minutes, 5 to 30 minutes, 5 to 10 minutes, etc.)).

In some embodiments, the lysis mixture is heated during the second time period with mixing. In certain embodiments, the mixing is at about 500 to 2000 rpm (e.g., 500 rpm, 600 rpm, 700 rpm, 800 rpm, 900 rpm, 1000 rpm, 1100 rpm, 1200 rpm, 1300 rpm, 1400 rpm, 1500 rpm, 1600 rpm, 1700 rpm, 1800 rpm, 1900 rpm, 2000 rpm, or ranges therebetween (e.g., 800 to 1500 rpm, etc.)).

In some embodiments, the nucleic acid comprises DNA. In certain embodiments, the nucleic acid comprises genomic DNA.

In some embodiments, the capture moiety is biotin. In certain embodiments, the solid particle is linked to streptavidin. In certain embodiments, the solid particle is a paramagnetic bead.

In some embodiments, the lysis mixture is incubated with the capture oligonucleotide before the solid particle is added to the lysis mixture. In certain embodiments, the solid particle and the oligonucleotide are added to the lysis mixture at the same time.

In some embodiments, the hybridization buffer contains sodium. In certain embodiments, the hybridization buffer comprises magnesium. In certain embodiments, the hybridization buffer comprises sodium and magnesium.

In some embodiments, the hybridization sample solution comprises about 200 to 1000 mM NaCl (e.g., 200 mM, 300 mM, 400 mM, 500 mM, 600 mM, 700 mM, 800 mM, 900 mM, 1000 mM, or ranges therebetween (e.g., 700 to 1000 mM, 400 to 600 mM, 400 to 500 mM, etc.)).

In some embodiments, the hybridization sample solution comprises about 10 to 200 mM MgCl₂ (e.g., 10 mM, 20 mM 50 mM, 75 mM, 100 mM 125 mM, 150 mM, 175 mM, 200 mM, or ranges therebetween (e.g., 50 to 200 mM, 25 to 75 mM, 50 to 100 mM, 50 to 75 mM, etc.)). In one aspect, a method for detecting one or more target nucleic acids in a biological sample which may contain a pathogen is provided.

In some embodiments, the method comprises mixing the sample with a thinning solution comprising SDS and proteinase K to form a lysis mixture; incubating the lysis mixture with a solid particle and at least one capture oligonucleotide, wherein the at least one capture oligonucleotide comprises a sequence which is complementary to a pathogen polynucleotide which comprises the target nucleic acid and the capture oligonucleotide hybridizes to the pathogen polynucleotide to form a particle complex comprising the solid particle, the capture oligonucleotide and the pathogen polynucleotide; washing the particle complex and eluting the pathogen polynucleotide from the particle complex to form a sample eluate; and using at least a portion of the sample eluate to a detection assay that can detect the target nucleic acid. In some embodiments, the method further comprises, further comprising transferring the at least a portion of the sample eluate to a tube containing a PCR reaction mix and performing a PCR reaction.

In some embodiments, the at least one capture oligonucleotide is complementary to a portion of the one or more target nucleic acids. In certain embodiments, the at least one capture oligonucleotide is complementary to a portion of the pathogen genomic DNA which is not part of the one or more target nucleic acids.

In some embodiments, the one or more target nucleic acid sequences comprises a MTBC IS6110 sequence. In certain embodiments, the at least one capture oligonucleotide comprises an oligonucleotide which is complementary to a MTBC IS6110 sequence. In certain embodiments, the at least one capture comprises the nucleotide sequence of SEQ ID NO:7 or SEQ ID NO:8. In some embodiments, the one or more target nucleic acid sequence comprises a MTBC senX3-regX3 sequence. In certain embodiments, the at least one capture oligonucleotide comprises an oligonucleotide which is complementary to the MTBC senX3-regX3 sequence. In certain embodiments, the at least one capture oligonucleotide comprises an oligonucleotide which comprises the nucleotide sequence of SEQ ID NO:9, SEQ ID NO:10 or SEQ ID NO:29. In some embodiments, the PCR reaction forward and reverse primers are complementary to a MTBC 156110 sequence. In certain embodiments, the PCR reaction forward primer comprises SEQ ID NO:16. In certain embodiments, the PCR reaction reverse primer comprises SEQ ID NO:17.

In some embodiments, the PCR reaction forward and reverse primers are complementary to a MTBC senX3-regX3 sequence. In certain embodiments, the PCR reaction forward primer comprises SEQ ID NO:19. In certain embodiments, the PCR reaction reverse primer comprises SEQ ID NO:20.

In one aspect, a method for detecting a M. tuberculosis nucleic acid in a sample from a subject is provided.

In some embodiments, the method comprises incubating the sample with a first primer pair which hybridizes to the senX3-regX3 intergenic region and a second primer pair which hybridizes to an IS6110 region.

In some embodiments, the first primer pair comprises a first senX3-regX3 primer comprising SEQ ID NO:19 and a second senX3-regX3 primer comprising SEQ ID NO:20.

In some embodiments, the second primer pair comprises a first 156110 primer comprising SEQ ID NO:16 and a second 156110 primer comprising SEQ ID NO:17.

In some embodiments, the sample is a sputum sample, wherein the sputum sample is treated with a lysis solution comprising SDS and PK.

In one aspect, a kit for detection of at least one target nucleic acid is provided wherein the kit comprises a collection cup and a sample lysis buffer and wherein the sample lysis buffer comprises SDS and PK.

In some embodiments, the kit further comprises a sample hybridization buffer, one or more capture probes each coupled to a capture moiety, and a plurality of solid particles wherein the solid particles bind to the capture moiety and the one or more capture probes comprises an oligonucleotide which is complementary to a polynucleotide which comprises the at least one target nucleic acid.

In some embodiments, the at least one target nucleic acid is a MTBC IS6110 sequence and/or a MTBC senX3-regX3 sequence.

In some embodiments, the kit further comprises a PCR reaction mix and at least one forward and reverse primer pair which is complementary to the at least one target nucleic acid.
In some embodiments, the at least one forward and reverse primer pair comprises SEQ ID NO:16 and SEQ ID NO:17. In certain embodiments, the at least one forward and reverse primer pair comprises SEQ ID NO:19 and SEQ ID NO:20. In certain embodiments, the at least one forward and reverse primer pair comprises SEQ ID NOs:16, 17, 19 and 20.
In some embodiments, the kit further comprises at least one PCR oligonucleotide probe. In certain embodiments, the at least one PCR oligonucleotide probe comprises SEQ ID NO:18 and/or SEQ ID NO:21.

### BRIEF DESCRIPTION OF DRAWINGS

FIGS. 1A and 1B illustrate results of qPCR reactions performed to test the effects of DNase I treatment of samples. FIG. 1A: 156110 qPCR assay. Standard curve of 500,000; 50,000; 5000, 500, 50, and 5 MTB H37Rv genomic DNA copies in triplicate. FIG. 1B: Box and whisker plot.

FIG. 2 illustrates a SDS/Proteinase K sputum thinning and sequence specific capture work flow.

FIGS. 3A-3C illustrate sputum thinning and DNA extraction methods and effects on qPCR. FIG. 3A illustrates bulk capture and sequence-specific capture methods. FIGS. 3B and 3C show the amount of co-extracted human genomic DNA (FIG. 3B) and MTB IS6110 yield (FIG. 3C) and.

FIG. 4 illustrates results of a qPCR reaction using samples prepared by Sputum processing with guanidinium hydrochloride.

FIG. 5 illustrates results of multiplexed qPCR to detect IS6110, senX3-regX3 and cotJC.

FIG. 6 illustrates MTB DNA yield is similar between buffer and 3 sputum specimens. Box and whisker plot of MTB yield from different specimen types: buffer, sputum 1, sputum 2, sputum 3, and all sputum results combined. Center lines show the medians; box limits indicate the 25th and 75th percentiles as determined by R software; whiskers extend to minimum and maximum values; crosses represent sample means; vertical dark gray bars indicate 95% confidence intervals of the means; data points are plotted as open circles.

FIG. 7 illustrates a schematic of the study one protocol for sample testing. SSM−=concentrated sputum smear microscopy negative; SSM+=concentrated sputum smear microscopy positive; C+=culture positive; C−=culture negative; CXR+=chest X-ray positive.

FIG. 8 illustrates distribution of genomic copies extracted from smear positive, scanty smear positive and smear negative samples. (A) Standard curve of senX3-regX3 log copy number 106-10 copies of 7 extractions combined (118 data points). Y=−3.42X+35.47. (95% CI of slope: −3.35, −3.48) R2=0.989. (B) Percentage of samples in different copy number distribution. SSM−=sputum smear microscopy negative (N=27); SCA=scanty sputum smear positive (N=19); SSM+=sputum smear microscopy positive (N=20). Numbers at top of bars indicate percentage of positive specimen in each category. Numbers at bottom of bar indicate number of positive specimen in each category.

FIG. 9 illustrates Bacillus spores are inefficiently lysed by heat step. (A) Standard curve of 1,000,000, 100,000, 10,000, 1,000, 100 and 10 cotJC gBlock copies in triplicate. Red curves are extracted DNA in triplicate. (B) cotJC qPCR assay is linear. Equation of line: y=−3.74x+37.96; (95% CI slope −3.86, −3.62) R2=0.996, qPCR efficiency=85.1%. (C) Box and whisker plot demonstrating that sample prep method did not sufficiently liberate B. atrophaeus spore DNA. Center lines show the medians; box limits indicate the 25th and 75^{th} percentiles as determined by R software; whiskers extend to minimum and maximum values; crosses represent sample means; vertical dark gray bars indicate 95% confidence intervals of the means; data points are plotted as open circles. Samples indicated by: Heat=heat lysis; GO=GeneOhm kit; Sp.Cap=specific capture protocol; GO.Sp.Cap=GeneOhm lysis followed by specific capture protocol. n=6, 6, 5, and 6 sample points, respectively.

FIG. 10 illustrates a schematic of the study two protocol for sample testing. SSM−=concentrated sputum smear microscopy negative; SSM+=concentrated sputum smear microscopy positive; C+=culture positive.

FIG. 11 illustrates amplification of 1 million genomic copies per reaction 4 NTM species with the original and optimized senX3-regX3 primer sets. Optimized primers show no amplification.

FIG. 12 illustrates standard curves of senX3-regX3 original and optimized primer sets with 50,000 to 5 M. tuberculosis genomic DNA copies. Optimized primers give ˜3 Cq improvement in performance.

### DEFINITIONS

Terms and abbreviations not defined should be accorded their ordinary meaning as used in the art. However, to facilitate an understanding of the present technology, a number of terms and phrases are defined below. Additional definitions are set forth throughout the detailed description.

As used herein, the singular forms “a,” “an,” and “the” encompass plural referents unless the context clearly dictates otherwise. Thus, for example, reference to “a protein” includes a plurality of such proteins and reference to “the formulation” includes reference to one or more formulations and equivalents thereof known to those skilled in the art, and so forth.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed by this disclosure. The upper and lower limits of these smaller ranges may independently be included or excluded in the range, and each range where either, neither or both limits are included in the smaller ranges is also encompassed by this disclosure, subject to any specifically excluded limit in the stated range. Where the stated range includes one or both of the limits, ranges excluding either or both of those included limits are also within the scope of this disclosure. For example, if a range of 5 to 10 minutes is stated, it is intended that 6 min., 7 min., 8 min., and 9 min. are also explicitly disclosed, as well as the range of values greater than or equal to 5 min. and the range of values less than or equal to 10 min.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

“Detection” of a target nucleic acid or analyte refers to determining the presence or the absence of the nucleic acid or analyte in a sample, where absence refers to a zero level or an undetectable level (e.g., at or beneath the background or noise).

As used herein, the term “sample” is used in the broadest sense. In one sense, a sample refers to a specimen obtained from any source, such as biological and environmental samples. Biological samples may be obtained from animals (including humans) and encompass fluids, solids, tissues, and gases. Samples may be obtained from a biological organism, a tissue, cell, cell culture medium, or any medium suitable for mimicking biological conditions, or from the environment. Non-limiting examples include bronchoalveolar lavage fluid, bronchial aspirates, lung biopsies, post-mortem tissue specimens, sputum, saliva, gingival secretions, cerebrospinal fluid, gastrointestinal fluid, mucous, urogenital secretions, synovial fluid, blood, serum, plasma, urine, cystic fluid, lymph fluid, ascites, pleural effusion, interstitial fluid, intracellular fluid, ocular fluids, seminal fluid, mammary secretions, vitreal fluid, nasal secretions, throat or nasal materials, pleural effusion, water, soil, biological waste, cell culture media, or any other fluid or solid media. In some embodiments, bacterial agents are contained in serum, whole blood, bronchoalveolar lavage fluid, bronchial aspirates, plasma, sputum, or nasal secretions. This also includes experimentally separated fractions of all of the preceding. For example, a blood sample can be fractionated into serum, plasma, or into fractions containing particular types of blood cells, such as red blood cells or white blood cells (leukocytes). In some embodiments, a sample can be a combination of samples from an individual, such as a combination of a tissue and fluid sample. The term “sample” may also include materials containing homogenized solid material, such as from a stool sample, a tissue sample, or a tissue biopsy; and materials derived from a tissue culture or a cell culture. Any suitable methods for obtaining a sample can be employed; exemplary methods include, e.g., phlebotomy, swab, and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection, bladder wash, smear, or ductal lavage. A sample obtained or derived from an individual includes any such sample that has been processed in any suitable manner (e.g., filtered, diluted, pooled, fractionated, concentrated, etc.) after being obtained from the individual.

The phrase “nucleic acid” or “polynucleotide” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

“Primers” refer to single-stranded oligonucleotides which are complementary to sequence portions on a template nucleic acid molecule separated by a variable number of nucleotides. Primers annealed to the template nucleic acid can be extended by covalent bonding of nucleotide monomers during amplification or polymerization of a nucleic acid molecule catalyzed by the thermostable polymerases. Typically, primers are from 12 to 35 nucleotides in length and are preferably from 15 to 20 nucleotides in length. Primers are designed from known parts of the template, one complementary to each strand of the double strand of the template nucleic acid molecule, lying on opposite sides of the region to be synthesized. Primers can be designed and synthetically prepared as is well known in the art.

The term used herein “forward primer” means a primer complementary to a strand of a nucleic acid sequence aligned in a 3′ to 5′ direction. The “reverse primer” has a complementary sequence to the other strand of the nucleic acid sequence.

“Template” as used herein refers to a double-stranded or single-stranded nucleic acid molecule, which serves a substrate for nucleic acid synthesis. In the case of a double-stranded DNA molecule, denaturation of its strands to form a first and a second strand is performed before these molecules may be used as substrates for nucleic acid synthesis. A primer, complementary to a portion of a single-stranded nucleic acid molecule serving as the temple template is hybridized under appropriate conditions and an appropriate polymerase may then synthesize a molecule complementary to the template or a portion thereof. The newly synthesized molecule may be equal or shorter in length than the original template.

A “target” or “target nucleic acid” refers to a single or double stranded polynucleotide sequence sought to be copied or amplified in a reaction which includes a polymerase and an oligonucleotide primer. A target nucleic acid may be genomic DNA or transcribed region of nucleic acid, the ends of which are base-complementary (with proper orientation) to primers included in a complete set of PCR reagents.

The term “hybridize” as used herein refers to the binding, duplexing, or hybridizing of a nucleic acid molecule preferentially to a particular nucleotide sequence. Hybridization generally involves the formation of hydrogen bonds between two single strands of a polynucleotide.

The term “complementary” as used herein refers to the capacity for precise pairing between two nucleotides; i.e., if a nucleotide at a given position of a nucleic acid is capable of hydrogen bonding (e.g., via standard Watson-Crick base pairing and Hoogsteen-type hydrogen bonding) with a nucleotide of another nucleic acid to form a canonical base pair, then the two nucleic acids are considered to be complementary to one another at that position. Complementarity between two single-stranded nucleic acid molecules may be “partial.” Complementarity is “complete,” fully,” or “100%” when there are no mismatches between the two single-stranded nucleotide sequences. “100% complementarity along the full length of the sequences” indicates that there are no mismatches between two nucleic acid strands which can hybridize and which are of identical length.

The term “oligonucleotide” as used herein refers to a sequence of nucleotide monomers, each bound to an adjacent nucleotide monomer by a covalent bond. An “oligonucleotide” may also include a non-nucleotide subunit or a nucleotide analog within the sequence of nucleotide monomers wherein the non-nucleotide subunit or nucleotide analog is bound to an adjacent subunit, analog or nucleotide by a covalent bond. The covalent bond between two adjacent nucleotide monomers in an oligonucleotide is a phosphodiester bond.

The term “dried” herein refers to a composition which has a water content of less than about 10%, 8%, 5%, 4%, 3%, 2%, 1% or 0.5%.

The “polymerase chain reaction” (“PCR”) is a reaction in which replicate copies are made of a target polynucleotide using one or more primers, and a catalyst of polymerization, such as a DNA polymerase, and particularly a thermally stable polymerase enzyme. Generally, PCR involves repeatedly performing a “cycle” of three steps: “melting,” in which the temperature is adjusted such that the DNA dissociates to single strands, “annealing,” in which the temperature is adjusted such that oligonucleotide primers are permitted to match their complementary base sequence using base pair recognition to form a duplex at one end of the span of polynucleotide to be amplified; and “extension” or “synthesis,” which may occur at the same temperature as annealing, or in which the temperature is adjusted to a slightly higher and more optimum temperature, such that oligonucleotides that have formed a duplex are elongated with a DNA polymerase. This cycle is then repeated until the desired amount of amplified polynucleotide is obtained. Methods for PCR amplification are taught, for example, in U.S. Pat. Nos. 4,683,195 and 4,683,202.

“Specificity” in primer extension or PCR amplification refers to the generation of a single, “specific” PCR product with the size and sequence predicted from the sequences of the primers and the genomic or transcribed region of nucleic acid to which the primers were designed to anneal in a base-complementary manner. “Nonspecific” PCR product has a size or sequence different from such prediction.

“Primer extension assay” refers to an in vitro method wherein a primer hybridized to a complementary sequence part of a single-stranded nucleic acid template molecule is extended by sequential covalent bonding of nucleotides to the 3′ end of the primer forming a new DNA molecule complementary to the DNA template molecule. The primer extension method transforms a single-stranded nucleic acid template into a partially or completely double-stranded nucleic acid molecule. The primer extension method as used herein is a single step nucleic synthesis process without amplification of the copy number of the template nucleic acid molecule.

### DETAILED DESCRIPTION

This disclosure relates generally to methods and kits useful for preparing samples, extracting nucleic acids from samples (e.g., biological samples), and/or detecting nucleic acids (e.g., pathogen nucleic acids) in samples (e.g., samples obtained from a subject). In particular, compositions, kits, and methods are provided comprising detergents and proteinases to treat biological samples prior to extraction of nucleic acids. Also described is use of cations for improved efficiency of nucleic acid hybridization. The prepared nucleic acid is suitable for PCR assays including those described for detection of Mycobacterium tuberculosis.

Several exemplary embodiments are described herein; however, this disclosure is not limited to the particular embodiments described. These embodiments may take many different forms, and/or portions of various embodiments combined, and should not be construed as limited to those embodiments explicitly set forth herein. Rather, these embodiments are provided so that this disclosure will be thorough and complete, and will fully convey the scope of the present disclosure to those skilled in the art. It is also to be understood that the terminology used herein is for the purpose of describing particular embodiments only, and is not intended to be limiting.

### Compositions and Methods for Preparing and Detecting Pathogenic Nucleic Acids

The present disclosure provides methods, kits, and compositions for preparing nucleic biological samples for the extraction of nucleic acids (e.g., target nucleic acids) from the sample. Methods, kits, and compositions are also provided for the downstream extraction and/or analysis of the nucleic acids (e.g., target nucleic acids). The methods and compositions are particularly useful for treating sputum samples which are often used for diagnosing Mycobacterial infections. Nevertheless, the disclosure can be applied to treatment of any biological or environmental sample for the preparation of nucleic acids that can be used in various nucleic acid detection assays.

As described below, in experiments conducted during development of embodiments herein human genomic DNA was identified as a key quantitative PCR (qPCR) inhibitor present in sputum, and sample preparation methods were developed that substantially eliminates it from the amplification reaction. A MTB screening assay was designed with a novel sample preparation method that achieves a limit of detection (LOD) of 20 cfu/ml which is more sensitive than the current NATs and approaches that of culture. Embodiments of this assay find use in the analysis and detection of other nucleic acids as well.

Also developed was a multiplexed qPCR assay targeting two Mycobacterium tuberculosis complex (MTBC)-specific loci to increase sensitivity and minimize the likelihood of false negatives due to target gene mutation or deletion. It includes the potentially multi-copy insertion sequence IS6110 (Thierry et al., 1990, J Clin Microbiol, 28:2668-2673; incorporated by reference in its entirety), and the highly conserved single-copy backup target senX3-regX3, a mycobacterial two-component regulatory operon critical for MTB virulence (Rifat and Karakousis, 2014, Microbiology, 160:1125-1133; incorporated by reference in its entirety). Amplification of the cotJC gene in Bacillus atrophaeus spores, which are added to the raw sputum, serves as a control for adequate processing of the target bacteria and to monitor qPCR inhibitors. The clinical performance of the test was verified with 60 sputum specimens collected from symptomatic TB patients with our test displaying 96% sensitivity and 100% specificity compared to the GeneXpert® MTB/RIF Assay.

### Identification of Inhibitory Components in Extracted Nucleic Acid Preparations

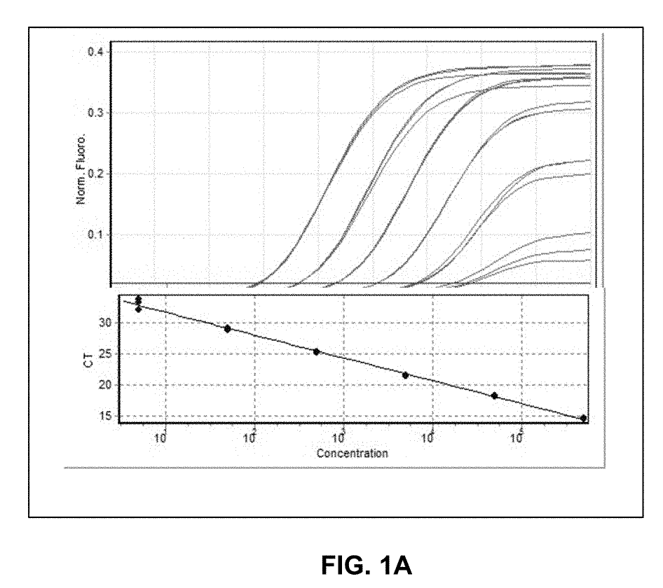

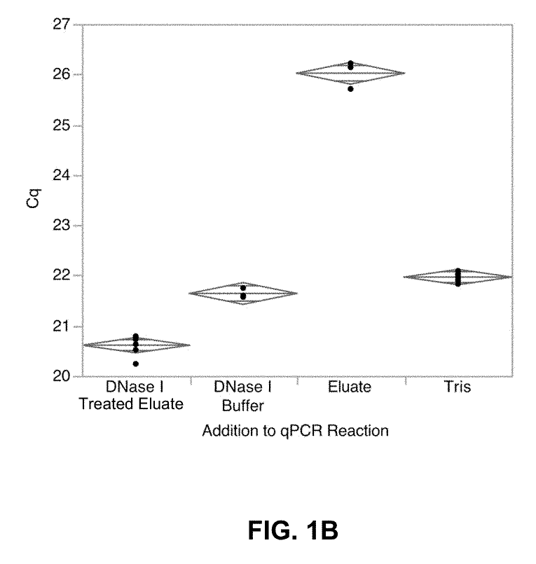

The Mycobacterium tuberculosis complex specific potentially multi-copy insertion sequence IS6110 (Thierry et al., 1990, J Clin Microbiol, 28:2668-2673; incorporated by reference in its entirety) as the genetic target for the qPCR assay to determine the efficacy of the MTB DNA extraction protocol from sputum. For the present studies, primers and probes (Table 2; SEQ ID Nos: 13-28) were designed to have 100% sequence identity to all complete MTBC species sequences available in the NCBI database. The assay was demonstrated to be linear across at least 6 logs of concentration from 500,000 to 5 copies (FIG. 1a) and can detect as few as 1 MTB H37Rv genome 6 out of 6 times.

Multiple investigators have reported that silica-based extraction methods such as the Boom method (Boom et al., 1990, J Clin Microbiol, 28:495-503; incorporated by reference in its entirety) yield MTB DNA with less PCR inhibition than alternative methods (Leung et al., 2011, J Clin Microbiol, 49:2509-2515; Suffys et al., 2001, Mem Inst Oswaldo Cruz, 96:1137-1139; van Doorn et al., 2006, Eur J Clin Microbiol Infect Dis, 25:673-675; incorporated by reference in their entireties). Dynabeads® SILANE Genomic DNA Kit (Life Technologies, Carlsbad, Calif.) protocol is designed to extract genomic DNA from 350 μl whole blood using 50 μl paramagnetic particles (PMP), but input volume can be adjusted to suit specific experimental needs. Bulk DNA extraction from sputum using Dynabeads® SILANE Genomic DNA Kit was evaluated in the presently described studies. Initial studies were performed with 350 μl contrived TB-positive sputum specimens with the intention of ultimately scaling up the sample volume to 1 ml. However, in extracting MTB DNA using this system, it was found that the subsequent qPCR results differed markedly between independent sputum samples. By amplifying 10-fold dilutions of the eluted DNA and quantifying the yield using a standard curve derived from serial dilutions of MTB H37Rv genomic DNA, it was determined that the MTB DNA yield from the Dynabeads® extraction was similar between samples, but qPCR inhibition varied dramatically (data not shown). In order to identify the potential source of inhibition, 7 sputum samples were evaluated for: appearance (bloody, mucopurulent, etc.), density, viscosity, protein levels and human genomic DNA levels and compared these results to qPCR inhibition. It was found that, of these variables, only human genomic DNA (gDNA) content correlated with inhibition (R²=0.985, Pearson correlation test). Three of the 7 samples displayed qPCR inhibition of greater than 1 Cq, and these 3 samples also had high levels of co-extracted human gDNA as measured by copy number of the single copy gene for β-globulin (data not shown). The other four sputa tested had much lower β-globulin copy number in the eluted sample and no evidence of qPCR inhibition.

Studies were then performed to confirm that the co-extraction of high amounts of human gDNA was the source of inhibition in the samples by treating sputum-derived DNA eluates with Turbo DNase I (Example 2). After treatment and heat-inactivating the DNase I, the eluates were added to tubes containing buffer only or a PCR reaction mix containing 5000 genomic copies of H37Rv genomic DNA. Relief of inhibition was observed in multiple specimens that had been treated with active DNase I (FIG. 1B). Analysis of the data indicates that inhibition by genomic DNA leads to more than a 10-fold under-quantification of the MTB bacterial load in a sample. The data also indicate that human genomic DNA in sputum is a frequent and potent qPCR inhibitor.

### Development of a Sequence Specific Capture Method

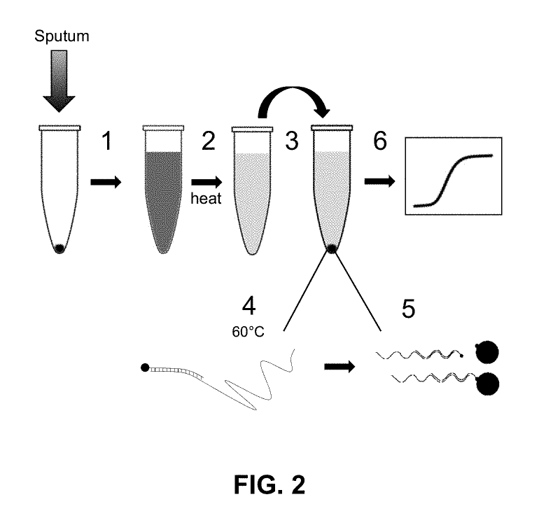

With the discovery that genomic DNA in a biologic sample significantly inhibits subsequent PCR amplification of a target sequence, a sequence-specific capture method was devised to partially remove genomic DNA in a sample without affecting the sensitivity of the reaction. To limit the co-extraction of human DNA from sputum specimens as observed with bulk DNA extraction, a protocol was developed to selectively purify mycobacterial DNA using sequence specific capture. An exemplary protocol is divided into two phases: 1) sputum thinning and pathogen lysis (steps 1-3) and 2) pathogen DNA capture (steps 4-6). The steps are further illustrated in FIG. 2: 1. Sputum is added to sputum thinning tube containing dried reagents represented as a black dot at bottom of tube; 2. Sputum is thinned, organisms are lysed, and double-stranded DNA is melted; 3. Thinned sputum is transferred to hybridization tube containing dried reagents represented as a black dot at bottom of tube; 4. Within the hybridization tube, the oligonucleotides (capture probes; Table 2 below) are hybridized to denatured DNA target at an appropriate temperature; 5. Streptavidin-coated PMPs form a complex with the capture probes to form a PMP-capture probe-DNA complex; 6. The DNA is eluted from the PMPs and subsequently amplified and detected using qPCR. The sputum is thinned to a pipettable consistency and simultaneously sterilized specimens with minimum operator steps. The process involves treating the sputum sample with a proteinase and an ionic detergent (Example 3). The detergent acts as an emulsifier breaking up mucoid complexes by unfolding proteins, and proteinase facilitates sputum thinning by digesting the unfolded proteins. Subsequent heating to a high temperature, e.g., 85° C. to 98° C., decontaminates the specimen by killing MTB and other flora present, while simultaneously denaturing the PK and initiating the specific capture reaction by melting the double stranded DNA in the specimen. Alternative protocols to the one set forth above, protocols comprising only a portion of the steps above, combination of the protocol and/or steps of the protocol with other method steps, and/or alterations to the above protocol are within the scope herein. In some embodiments, the above steps, or the steps of other protocols within the scope herein, are performed in one or more containers (e.g., 1, 2, 3, 4, 5, 6, etc.). In some embodiments, sample and reagents are maintained in a single vessel for multiple steps. In some embodiments, sample and reagents are transferred between two or more vessels during or between steps. In some embodiments, steps 1-3, the thinning and lysing steps (or analogous steps in a separate protocol) are performed in a first vessel. In some embodiments, thinning and lysing reagents are provided with the first vessel (e.g., dried to a wall of) and the biological sample is added to the vessel. In some embodiments, subsequent steps are also performed in the first vessel. In other embodiments, subsequent steps are performed in one or more additional vessels (e.g., with liquid or dried reagents contained therein).

TABLE 1

| Genetic | SEQ ID | |
|---|---|---|
| Target | NO | Oligonucleotide Sequence |
| MTB | 1 | 5′-Biotin-AAAAACGAACGGCTGATGACCAAACTC-3′ |
| IS6110* | | |
| MTB | 2 | 5′-Biotin-AAAAAGGAGGTGGCCATCGTGGAAG-3′ |
| IS6110* | | |
| MTB senX3- | 3 | 5′-Biotin- AAAAACAGTGTGTTGATTGTGGAGGACGAG-3′ |
| regX3 | | |
| MTB senX3- | 4 | 5′-Biotin-AAAAAGTGGAATCAAAGCCCTCCTTGCG-3′ |
| regX3 | | |
| MTB senX3- | 30 | 5′Biotin-AAAAACAGAGCGTAGCGATGAGGTGGG-3′ |
| regX3 | | |
| BA cotJC | 5 | 5′-Biotin- |
| | | AAAAACAAATACTTAATCGAGCAATATGGCGGCG-3′ |
| BA cotJC | 6 | 5′Biotin- |
| | | AAAAAGCTTATACACCATCGTCGCAATCATCTCC-3′ |
| MTB | 7 | 5′-CGAACGGCTGATGACCAAACTC-3′ |
| IS6110* | | |
| MTB | 8 | 5′-GGAGGTGGCCATCGTGGAAG-3′ |
| IS6110* | | |
| MTB senX3- | 9 | 5′-CAGTGTGTTGATTGTGGAGGACGAG-3′ |
| regX3 | | |
| MTB senX3- | 10 | 5′-GTGGAATCAAAGCCCTCCTTGCG-3′ |
| regX3 | | |
| MTB senX3- | 29 | 5′-CAGAGCGTAGCGATGAGGTGGG-3′ |
| regX3 | | |
| BA cotJC | 11 | 5′-CAAATACTTAATCGAGCAATATGGCGGCG-3′ |
| BA cotJC | 12 | 5′-GCTTATACACCATCGTCGCAATCATCTCC-3′ |

*From Mangiapan et al., 1996, J Clin Microbiol 34:1209-1215.

In some embodiments, a method for preparing nucleic acid from a sputum sample (or other biological sample) is provided in which the sputum is not diluted. In some embodiments, the sputum (or other biological sample) is diluted by the addition of liquid reagents with a combined volume of less than 50% (e.g., 45%, 40%, 35%, 30%, 25%, 20%, 15%, 10%, 9%, 8%, 7%, 6%, 5%, 4%, 3%, 2%, 1%, 0.5%, 0.2%, 0.1%, or less) of the volume of the original sample. In some embodiments, a method for preparing nucleic acid from a sputum sample is provided wherein the method comprises mixing the sputum with a protease and a detergent. The protease can be any protease readily available, however, in some embodiments, the protease is proteinase K. The detergent can be a neutral or an anionic detergent. In some embodiments, the detergent is not a cationic detergent. Detergents useful in the method include but are not limited to SDS, sarkosyl, deoxycholate, cholate, CHAPSO and CHAPS. The sputum sample is incubated in the presence of at least the detergent and the proteinase at a temperature that allows the proteinase activity. The incubation is for a period of time sufficient to allow thinning of the sputum, for example, between 1 and 15 minutes (e.g., 1 minutes, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, or ranges therebetween (e.g., 5 to 15 minutes, 5 to 10 minutes, 5 to 20 minutes, 1 to 10 minutes, 1 to 9 minutes, 4 to 10 minutes, 7 to 10 minutes, etc.)). In some embodiments, the time period is greater (e.g., >10 minutes, >15 minutes, >20 minutes, >25 minutes, >30 minutes, >45 minutes, >60 minutes, etc.) when lower temperatures (e.g., <45° C., <40° C., <35° C., <30° C., or less) are used. In some embodiments, the time period is less (e.g., <10 minutes, <9 minutes, <8 minutes, <7 minutes, <6 minutes, <5 minutes, <4 minutes, <3 minutes, <2 minutes, <1 minute, or less) when higher temperatures (e.g., >40° C., >45° C., >50° C., >55° C., >60° C., >65° C., or more) are used. In some embodiments, the sample is treated with about 0.1 to 5% SDS (e.g., 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, or ranges therebetween (e.g., 1 to 2%, 0.5 to 1.5% or 0.75 to 1.5%, etc.)), and/or with about 0.1 to 5% ionic detergent (e.g., 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, or ranges therebetween (e.g., 1 to 2%, 0.5 to 1.5% or 0.75 to 1.5%, etc.)). In some embodiments, proteinase is added to the sample. For example, in some embodiments, a 1 ml sputum sample is treated with about 25 to 500 units or proteinase (e.g., 25 units, 50 units, 75 units, 100 units, 200 units, 300 units, 400 units, 500 units, or ranges therebetween (e.g., 50 to 150 units, 20 to 100 units, 30 to 80 units, 40 to 70 units, 50 to 80 units, etc.). In some embodiments, at least about 50 units of proteinase is used per 1 ml of sputum. In some embodiments, 1 unit or proteinase is an amount of enzyme that liberates 1 μmole of Folin-positive amino acid within one minute at 37° C. using hemoglobin as a substrate. In some embodiments, 0.1 to 5 mg (e.g., 0.1 mg, 0.25 mg, 0.5 mg, 0.75 mg, 1 mg, 2 mg, 3 mg, 4 mg, 5 mg, or ranges therebetween (e.g., 0.25 to 2.5 mg, 1 to 2 mg, or 1.25 to 1.75 mg, etc.)) of proteinase (e.g., proteinase K) is added.

In some embodiments, the sample is incubated with proteinase and/or detergent at a temperature which allows the proteinase to function such as between about 20° C. to 70° C. (e.g., 20° C., 25° C., 30° C., 35° C., 40° C., 45° C., 50° C., 55° C., 60° C., 65° C., 70° C., or ranges therebetween (e.g., 40° C. to 60° C., 25° C. to 55° C., 40° C. to 60° C., 45° C. to 55° C., 50° C. to 60° C., etc.)). In some embodiments, incubation occurs with or without mixing. In some embodiments, the sample is mixed during the time period of incubation.

In some embodiments, after incubating the sputum with the detergent and/or proteinase, the temperature of the solution is increased, for example, to about 80° C. to 98° C. (e.g., 80° C., 82° C., 84° C., 86° C., 88° C., 90° C., 92° C., 94° C., 96° C., 98° C., or ranges therebetween (e.g., 85° C. to 95° C., 90° C., to 98° C., etc.)). The sputum solution is incubated at the temperature for a time period of at least 1 minute (e.g., 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, or ranges therebetween (e.g., 5 to 10 minutes, 8 to 10 minutes, 5 to 15 minutes, at least 5 minutes, at least 10 minutes, etc.)). In some embodiments, the sputum solution is incubated at the temperature for a time period no longer than 30 minutes. In some embodiments, the increase in temperature serves to denature the proteinase and/or denature the helices of DNA in the treated sample.

In some embodiments, after treatment of the sample with detergent and proteinase, the thinned sputum is transferred to a fresh tube to generate an oligonucleotide binding buffer sample solution. In some embodiments, the fresh tube contains a dried composition which, when solubilized in the thinned sputum, forms a solution in which nucleic acid in the thinned sputum can hybridize to oligonucleotides in a sequence-specific manner. In alternative embodiments, the thinned sputum is transferred to a tube and mixed with components to generate a solution conducive to nucleotide strand hybridization. In other embodiments, components (e.g., dried and/or liquid) to generate a solution conducive to nucleotide strand hybridization are added to the treated sample, without changing tubes. In some embodiments, the oligonucleotide binding buffer sample solution containing the thinned sputum comprises one or more of the following ingredients: NaCl, buffer, divalent cation chelating agent, nonionic detergent, etc. In some embodiments, the binding buffer sample solution has a pH of 5.5 to 9.5 (e.g., 5.5, 6.0, 6.5, 7.0, 7.5, 8.0, 8.5, 9.0, 9.5, or pH ranges therebetween (e.g., 6.5 to 9.0, 7.0 to 8.0, 7.5 to 8.5, etc.)). In some embodiments, the buffer in the binding solution is present at a concentration of about 1 to 50 mM (e.g., 1 mM, 2 mM, 5 mM, 10 mM, 20 mM, 50 mM, or ranges therebetween (e.g., 5 to 15 mM, etc.)). In some embodiments, the buffer is 10 mM Tris. In some embodiments, the divalent cationic chelating agent, if present, is at a concentration of about 0.1 to 2.5 mM (e.g., 0.1 mM, 0.2 mM, 0.5 mM, 1.0 mM, 1.5 mM, 2.0 mM, 2.5 mM, oranges therebetween (e.g., 0.1 to 1.5 mM, 0.5 to 1 mM, etc.)). In some embodiments, the non-ionic detergent if present is at a concentration of 0.001% to 0.1% (e.g., 0.001%, 0.002%, 0.005%, 0.01%, 0.02%, 0.05%, 0.1%, or ranges therebetween (e.g., 0.005% to 0.01%, etc.)) of the oligonucleotide binding buffer sample solution. In some embodiments, the non-ionic detergent is TWEEN® 20 (polyethylene glycol sorbitan monolaurate).

In some embodiments, a dried oligonucleotide binding buffer prepared as described above is prepared in a specimen collection container rather than a tube used in the laboratory. In some embodiments, such a collection container is useful in a kit such that lysis and the preparation method can begin upon collection of the specimen.

In some embodiments, after generation of the oligonucleotide binding buffer sample solution which contains the thinned sputum, oligonucleotides (capture probes) are added which have a sequence specific to a target sequence. In some embodiments, the oligonucleotides are configured to specifically hybridize to sequences within or near one or more target sequences of the pathogen to be detected in the sputum. In some embodiments, one or more distinct oligonucleotides are added to the binding buffer sample solution, depending on the number of target sequences to be detected (e.g., in a later assay). In some embodiments, a capture probe is added which has a sequence that hybridizes to a housekeeping sequence or other control sequence that allows monitoring of the presence of non-pathogenic nucleic acids. In some embodiments, the oligonucleotides are bound to a capture moiety which can specifically bind to a capture agent (e.g., displayed on a solid particle or surface). In a preferred embodiment, the capture moiety is biotin (to form biotinylated oligonucleotides as capture probes), the capture agent is streptavidin, and the streptavidin is displayed on a paramagnetic particle (PMP). Other similar capture-moiety/capture-agent systems find use in embodiments herein, as readily understood by the ordinarily skilled artisan and commercially available. For example, in some embodiments, an oligonucleotide is coupled to a solid particle though a carbodiimide linkage. The capture moiety and capture agent interaction may be non-covalent, for example members of a binding pair, such as: antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, etc. In other embodiments, the capture moiety and capture agent interaction is a covalent binding, such as sulfhydryl/maleimide, sulfhydryl/haloacetyl derivative, amine/isotriocyanate, amine/succinimidyl ester, amine/sulfonyl halides, etc.

In an exemplary capture process, capture probes (e.g., comprising (i) an oligonucleotide that hybridizes to a target or target-approximate sequence and (ii) a capture moiety) are added to the prepared oligonucleotide binding buffer sample solution and nucleic acid present in the sample solution is incubated to allow hybridization of the capture probes to nucleic acids in the sample solution. In some embodiments, the time and temperature of this hybridization step depends on, e.g., the length and sequence of the capture probe and can be readily determined by a person having ordinary skill in the art. Next, solid particles (e.g., displaying a capture agent that is a complement to the capture moiety) are added to the sample solution, allowed to bind to the capture probes to form a bound capture complex that is washed and isolated from the bulk of the binding solution (e.g., by a magnetic rack for PMPs, by centrifugation for other solid particles, etc.). The bound capture complex comprises the PMP, capture probe and a nucleic acid which is complementary to the capture probe. The isolated capture complexes are resuspended in an elution buffer and heated to melt the capture probe duplexes and release from the complex nucleic acid that had been present in the thinned sputum. In some embodiments, the particle complexes are resuspended in a volume of solution which provides an optimal concentration of nucleic acid released from the capture probes and particles. For example, in some embodiments, the particle complexes are resuspended in a volume of about 5 to 100 μl (e.g., 5 μl, 10 μl, 15 μl, 20 μl, 25 μl, 30 μl, 35 μl, 40 μl, 45 μl, 50 μl, 60 μl, 70 μl, 80 μl, 90 μl, 100 μl, or ranges therebetween (e.g., 5 to 15 μl, 5 to 20 μl, 10 to 20 μl, 10 to 50 μl, 25 to 50 μl, etc.)). A person having ordinary skill in the art can readily determine the appropriate time and temperature for allowing melting of the capture probe duplexes and release of sample nucleic acid from the bound complexes while maintaining the integrity of the nucleic acid that had been in the thinned sputum. For example, the resuspended particles can be incubated at a temperature ranging from about 70° C. to 95° C., (e.g., 70° C., 75° C., 80° C., 85° C., 90° C., 95° C., or ranges therebetween (e.g., 70° C. to 80° C., 75° C. to 85° C., 70° C. to 75° C., 80° C. to 85° C., 90° C. to 95° C., etc.)). In some embodiments, the eluted DNA is subsequently amplified and detected using real-time PCR or other nucleic acid detection techniques including but not limited to isothermal amplification, including helicase dependent amplification, cross-primer amplification and DNA sequencing.

### Sequence Specific Capture Versus Silica Bulk DNA Capture

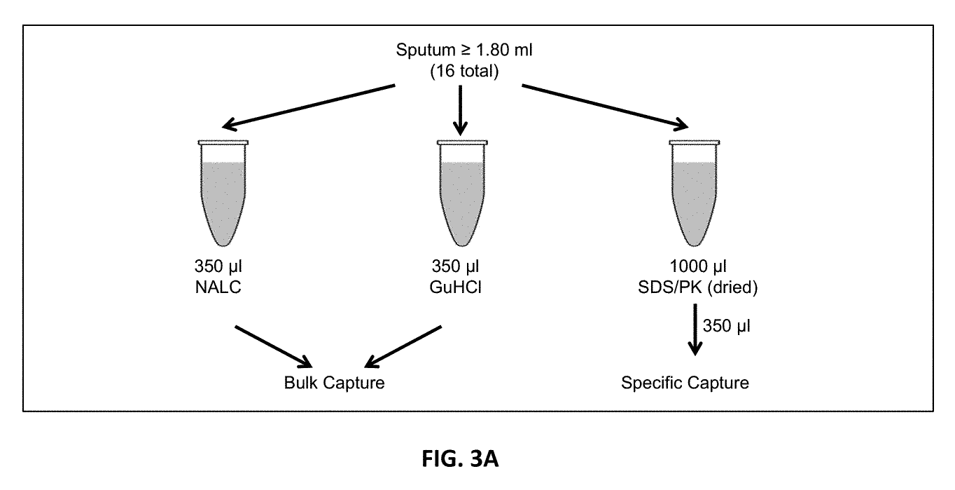

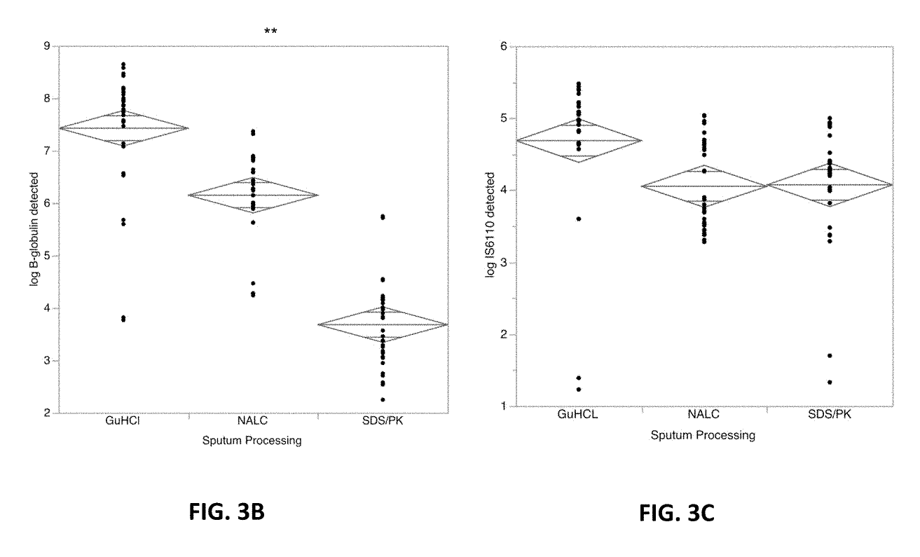

To demonstrate advantages of the sequence-specific capture method described above, quantitative PCR (qPCR) reactions were performed using template obtained through the sequence-specific capture method and compared to qPCR amplification using template prepared by one of two bulk DNA preparation methods: 1) N-acetyl-L-cysteine-sodium hydroxide (NALC), 2) guanidinium hydrochloride (GuHCl) which is the first step of the Dynabeads® SILANE Genomic DNA Kit (Life Technologies, Carlsbad, Calif.) (see Example 3). For this study (Example 4), contrived sputum samples were thinned and DNA was extracted as described above and in Example 3. MTB DNA yield was measured by quantitating IS6110 copies (FIG. 3C), human gDNA yield was measured by quantitating β-globulin copies (FIG. 3B), and qPCR inhibition was compared between the three strategies. SDS/PK treated sputum yielded the lowest levels of co-extracted human DNA while GuHCl treated sputum yielded the highest levels of co-extracted human DNA among the three DNA extraction methods tested, as reflected by the single-copy human gene β-globulin, as well as strongest assay inhibition.

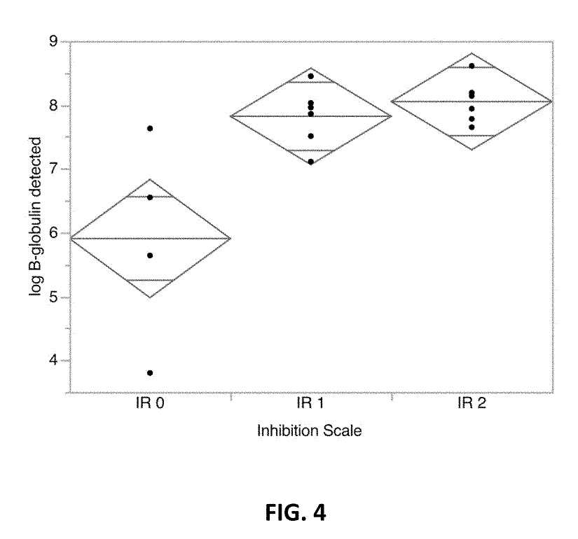

None of the NALC-treated or SDS/PK-treated samples demonstrated inhibition of the qPCR assays, however, 75% of the GuHCl treated specimens exhibited qPCR inhibition with the higher the amount of β-globulin co-extracted corresponding to higher qPCR inhibition (FIG. 4). A further analysis of the three sputum processing methods showed that while none of the NALC-treated samples demonstrated inhibition of the qPCR assays described in Example 4, use of an amount of eluate obtained from approximately 1 ml of sputum would result in quantities of genomic DNA in the sample high enough to inhibit qPCR reactions. However, the SDS/PK treated specimens average gDNA yield is still below the initial average NALC treated which seems to indicate that even with one ml of sputum and the entire elution being amplified, the SDS/PK samples would not be inhibited by co-extracted human gDNA. Due to the fact that specific capture based DNA extraction resulted in equivalent IS6110 copies (FIG. 3C), but significantly lower human gDNA (FIG. 3B), SDS/PK was selected as the preferred nucleic acid extraction strategy for the MTB diagnostic assay.

Multiplexed qPCR Assay

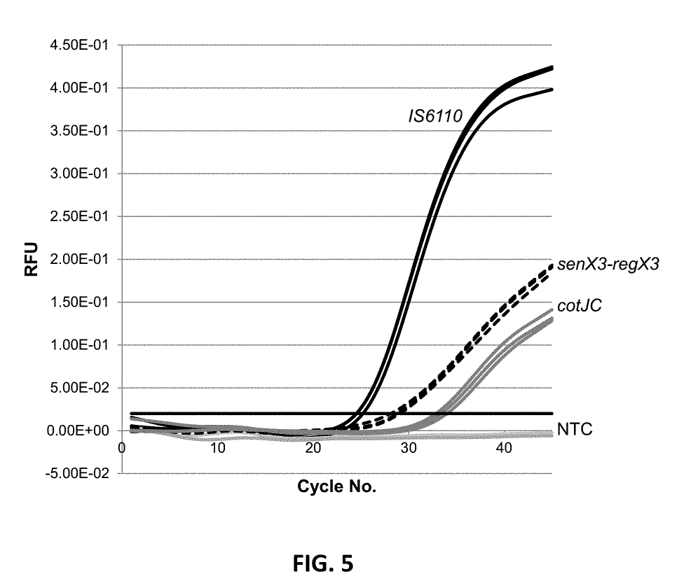

To test the SDS/PK sample preparation method with genuine TB-suspect specimens, a multiplex assay was first developed using contrived samples (Example 5). The multiplex assay was designed to detect all MTB strains by multiplexing the IS6110 assay with primers and probes specific to an intergenic region in the two-component regulatory operon senX3-regX3 (Rifat and Karakousis, 2014, Microbiology, 160:1125-1133). Both targets are MTB complex (MTBC) specific, but IS6110 is not present in some clinical strains (Lok et al., 2002, Emerg Infec Dis, 8:1310-1313), therefore multiplexing with another diagnostic amplicon will enhance sensitivity of detection. An amplicon of the Bacillus atrophaeus cotJC gene that encodes a spore coat composition polypeptide (Seyler et al, 1997, Mol Microbiol, 25:955-966) was also included to monitor the test for extraction, qPCR inhibitors, and amplification efficiency. Capture probes (Table 1) were designed for each specific target, tested in isolation and then in combination for optimal specific MTB DNA extraction (FIG. 5). Contrived sputum specimens of MTB H37Ra and B. atrophaeus spores were extracted using the SDS/PK sequence specific capture MTB DNA extraction method, and the multiplexed PCR assay was performed to confirm capture of multiple targets and assay performance (FIG. 5). The IS6110 amplicon had an average Cq of 24.8±0.4, the senX3-regX3 had an average Cq of 28.7±0.5 and the cotJC amplicon had an average Cq of 32.1±0.6.

### Laboratory Validation Study of Sputum Processing and Work Flow

Validation of the methods described above was carried out by processing and analyzing clinical specimens. Fifty-nine blinded samples from a hospital were processed using SDS/PK extraction and sequence-specific capture, then analyzed by multiplex qPCR to detect both IS6110 and senX3-regX3. The clinical samples included 27 positive specimens and 32 negative specimens. This validation study (Example 6) shows a sensitivity of about 89% and 96% for the senX3-regX3 and IS6110 assays, respectively, and 100% specificity.

A second clinical study was performed in which 94 clinical specimens from patients suspected of pulmonary TB were testes (Example 6). Smear and culture testing results were blinded and decoded after testing and the results confirmed extremely high overall assay sensitivity with regard to culture-positive specimens (97%, N=70) and specificity with regard to true negative specimens (100%, N=18). This population of sputum has historically been the most challenging to diagnose, and in this study, it was determined that more than half of the samples we tested in this group had less than 100 genomic copies of senX3-regX3 as normalized to MTB H37Rv genomic DNA. This emphasizes the requirement of a highly sensitive test that has minimal qPCR inhibition in order to efficiently detect MTB in smear negative specimens.

### Effects of Sodium and Magnesium

Hybridization in a nucleic acid diagnostic can be a rate limiting step which impacts efficiency of the diagnostic reaction. It follows that hybridization of the capture probe to nucleic acid in the thinned sample can also impact the efficiency of a diagnostic test which includes preparation of template nucleic acid from a sample and detection of target sequences in the sample. Improvements in probe/target interaction can be directly related to optimized extraction efficiency (copies recovered:total copies) of the specific capture, regardless of time.

As various studies were performed as described above to improve methods for extracting nucleic acids from a biological sample and reduce the presence of inhibitors in the eluates, observations were recorded which suggested that salts used during the processing may impact the efficiency and speed of the eluate preparation. Specifically, it was shown (Example 7 and Table 5) that adding a combination of sodium and magnesium cations to hybridization reaction between oligonucleotide capture probes and complementary nucleic acid present in a thinned sputum sample increases both the speed and efficiency of hybridization. As shown in Table 5, the presence of both sodium and magnesium during incubation of capture probes and thinned sputum resulted in more DNA capture during a 5 minute incubation than did the presence of only sodium during a 20 minute incubation.

In some embodiments, a method of processing a sample for a nucleic acid detection assay is provided comprising incubating the sample containing genomic DNA with a capture probe in the presence of both sodium and magnesium. In some embodiments, the sodium is in the form of NaCl. In certain embodiments, the magnesium is in the form of MgCl₂. In some embodiments, the solution containing the genomic DNA and capture probe contains between about 200 to 1000 mM, 200 to 750 mM, 400 to 600 mM, 300 to 600 mM, or 450 to 550 mM NaCl or contain 200 mM, 300 mM, 350 mM, 400 mM, 450 mM, 500 mM, 550 mM, 600 mM, 650 mM, 700 mM, 800 mM, 900 mM or 1000 mM NaCl. In some embodiments, the solution further comprises MgCl₂ at a concentration of about 10 to 400 mM, 25 to 400 mM, 25 to 200 mM, 25 to 100 mM, 25 to 75 mM, 40 to 60 mM, 10 to 100 mM or 10 to 75 mM MgCl₂, or comprises about 10 mM, 25 mM, 30 mM, 40 mM, 50 mM, 60 mM, 70 mM, 80 mM, 90 mM or 100 mM MgCl₂. In some embodiments, the solution containing the genomic DNA and capture probe is incubated with the NaCl and MgCl₂ for about 2 to 20 min, 2 to 15 min, 2 to 10 min, 2 to 8 min, 2 to 5 min, 5 to 10 min, 5 to 15 min or 5 to 20 min, or for about 2 min, 3 min, 4 min, 5 min, 6 min, 7 min, 8 min, 9 min, 10 min, 12 min, 15 min, 18 min or 20 min.

It is understood by the ordinarily skilled artisan that optimal amounts of sodium and magnesium ions depend on reaction conditions, including but not limited to, probe sequence, probe concentration, incubation temperature and incubation time, any of which can be readily and appropriately adjusted.

The present disclosure provides methods of sputum pre-treatment, extraction and amplification that, combined, address many of the challenges of MTB diagnosis. The pre-treatment method liquefies sputum and lyses pathogenic cells without the dilution associated with liquid solutions. It also results in a homogeneous distribution of pathogen DNA that eliminates potential sampling errors due to its buoyancy and tendency to clump and chain. The specific capture extraction method minimizes co-extraction of human DNA which is a major inhibitor of amplification. By combining IS6110, a potentially multi-copy target that is not present in all MTB strains, and senX3-regX3, a single-copy target in a genetic locus that is essential for virulence (Parish et al, 2003, Microbiol, 149:1423-1435; incorporated by reference in its entirety), an assay has been created that will detect all MTBC strains with high sensitivity. A preliminary estimate of the LOD is 20 cfu/ml for the combined assay which is more than 6-times lower than the LOD of the leading commercial test, which is reported as 131 cfu/ml (Helb et al., 2010, J Clin Microbiol, 48:229-237; incorporated by reference in its entirety).

In some embodiments, in order to achieve this high sensitivity, potential sputum-derived factors that could contribute to assay inhibition were methodically evaluated and led to the identification of human genomic DNA as a major direct contributor to MTBC-specific qPCR assay inhibition. High amounts of human genomic DNA could have a secondary technical effect of making the PMPs sticky, leading to greater co-extraction of inhibitors from either the biological specimen or DNA extraction solvents such as guanidinium or alcohols. Guanidinium hydrochloride treated sputum yielded the highest levels of co-extracted human DNA among the three DNA extraction methods tested, as reflected by the single-copy human gene β-globulin, as well as strongest assay inhibition.

By introducing a sample of 1 ml of whole sputum specimen into the tube containing dried sputum thinning reagent for sputum liquefaction and decontamination, a strategy was developed that mimics the processing of sputum for mycobacterial culture growth, but eliminates the cumbersome sample neutralization and centrifugation steps. This strategy also addresses the biohazard risk associated with inadequate management of infectious samples and the limitations of sample preparation methods to enhance the overall sensitivity of the assay. Sputum collection and manipulation from putative TB patients puts healthcare workers at risk for infection (Jensen et al., 2005, MMWR Recomm Rep, 54:1-141; Niemz and Boyle, 2012, Expert Rev Mol Diagn, 12:687-701; incorporated by reference in their entireties) and, for NAT testing in settings without biosafety precautions, it is necessary to completely sterilize the sample. This MTB screening assay is able to use a larger volume of sputum (e.g., 1 ml) which will be completely lysed and fully homogenized before the test aliquot is removed. This ameliorates the risk of sampling error and biosafety hazard to the operator. It is not reliant a BSL-3 facility thereby expanding its potential to be applied in limited-resource settings or even at the patient point-of-care, nor is it reliant on centrifugation, therefore improving sensitivity by using both intact and lysed mycobacterial DNA (Pathak et al, 2007, BMC Microbiol, 7:83; incorporated by reference in its entirety) that would otherwise be discarded in supernatants of centrifugation-based extraction methods. In some embodiments, by using paramagnetic particles (PMP), MTB DNA is extracted from a larger specimen volume, and the sputum-derived inhibitors are removed with simple wash steps. In some embodiments, a sputum processing protocol is utilized that is performed directly in the sputum collection cup, in order to minimize operator handling and potential for cross-contamination.

Experiments conducted during development of embodiments herein demonstrate use of specific capture based DNA extraction in other applications of clinical diagnostics and in other clinical sample types where target DNA may be underrepresented relative to a large pool of human DNA or other potential assay inhibitors. Compared to the two other common sputum processing and DNA extraction methods discussed here (NALC or GuHCl treatment with bulk DNA extraction), while specific capture based DNA extraction resulted in an equivalent level of MTB-specific IS6110 copies, there was a significant reduction (2.5 to 3.7 log copies β-globulin DNA) in co-eluted human genomic DNA. When tested on a panel of TB positive and negative clinical sputa, showed a sensitivity of 88.9-96.3% for senX3-regX3 and IS6110, respectively relative to Xpert® MTB/RIF, with 100% specificity in both targets.

The WHO Millennium Development Goal of stopping the increase of TB incidence and halving the TB-associated mortality by 2015 was achieved globally, but not in Africa (WHO, Global Tuberculosis Report 2015, W. H. Organization, Editor 2015, World Health Organization: Geneva, Switzerland; Raviglione et al., 2012, Lancet, 379:1902-1913; incorporated by reference in their entireties). The next goal set by WHO is to eliminate TB by 2050. This will require a reduction of TB of 16% per year compared to the current rate of 1% per year. In order to achieve these goals, major advances in diagnostics, vaccines and social policy are required (Wejse, 2015, Int J Infect Dis, 32:152-155; incorporated by reference in its entirety). Increasing diagnostic sensitivity is capable of dramatically reducing delays in diagnosis and treatment, and it has been estimated that every 10% increase in test sensitivity can reduce diagnostic delays experienced by patients by 3-5 days (Millen et al., 2008, PLoS One, 3:e1933; incorporated by reference in its entirety). However, without a combinatorial approach, the success of even a promising TB diagnostic test is not likely to impart a significant impact in high burden settings. In particular, a highly sensitive assay is required to reduce reliance on empiric treatment and improve detection in highly vulnerable populations such as children and people living with HIV. Specifically, this will require targeted development of diagnostic tests designed to address the challenges of MTB including the intrinsic properties of difficult clinical sample types that may limit diagnostic assay performance, and barriers of implementation in settings with high HIV and TB incidence.

### EXAMPLES

### Example 1

### Materials and Methods

### Bacterial Strains, Genomic and Plasmid DNA and Sample Sources

Two MTBC strains, Mycobacterium tuberculosis H37Ra and Mycobacterium bovis BCG were acquired from the American Type Culture Collection (ATCC; Manassas, Va.) and used as positive controls for assay development. Mycobacterial samples were sonicated to break up cell clumps prior to contriving sputum specimens as described by Helb et al. (2010, J Clin Microbiol, 48:229-237). Bacillus atrophaeus spores (MesaLabs; Lakewood, Colo.) were used as the process control. M. tuberculosis H37Rv TMC 303 genomic DNA (ATCC; Manassas, Va.) was used as the template for DNA-specific capture and real-time PCR assays. Genomic DNA from 6 Mycobacterium species: M. gordonae, M. intracellulare, M. terrae, M. malmoense, M. celatum, and M. abscessus (ATCC; Manassas, Va.) was used as the template for the specificity panel. The β-globulin plasmid used to generate a standard curve of the human single-copy β-globulin gene, HBB, (Abravaya et al, U.S. Pat. Pub. No. 2010/0081124), was constructed by cloning 476 bp of nucleotide 76-552 of the HBB sequence (GenBank Acc. No. KP309822) into the EcoR1 site of pGEM(R)-T Easy Vector (Promega; Madison, Wis.). Residual sputum specimens were obtained from TriCore Reference Laboratories (Albuquerque, N. Mex.) in frozen 1 ml aliquots.

### Quantitative PCR

A PCR master mix for a 25 μl total reaction volume (15 μl master mix and 10 μl of eluted DNA) consisted of: 0.2 mg/ml bovine serum albumin (BSA; Life Technologies Corporation; Grand Island, N.Y.), 0.2 mg/ml TWEEN® 20, 150 mM trehalose (Sigma; St. Louis, Mo.), 10% glycerol, 62.5 mM bicine pH 8 (Affymetrix; Santa Clara, Calif.), 135 mM potassium acetate pH 7.5 (Affymetrix; Santa Clara, Calif.), 1.5 mM manganese chloride (Sigma; St. Louis, Mo.), 0.325 mM each dNTP (Life Technologies Corporation; Grand Island, N.Y.), 3.75 U RMS Z05 DNA polymerase (Roche Molecular Systems, Inc., Branchburg, N.J.), and sequence-specific oligonucleotides (Table 1). For the IS6110 single-plex assays, 200 nM IS6110 F7, 200 nM IS6110 R10, and 300 nM IS6110 probe were used. For the β-globulin assays, 100 nM bgF, 100 nM bgR and 150 nM β-globulin probe were used. For the multiplexed MTB assay, 200 nM IS6110 F7, 200 nM IS6110 R10, 300 nM IS6110 probe, 300 nM senX3-regX3 F3, 200 nM senX3-regX3 R2, 200 nM senX3-regX3 probe 8, 100 nM cotJC F2, 100 nM cotJC R2 and 100 nM cotJC probe3. The primer and probe sequences are provided below in Table 2.

TABLE 2

| Genetic | Oligo | |
|---|---|---|
| Target | Name | Oligonucleotide sequence |
| Human β- | SEQ ID | 5′-GGCAGGTTGGTATCAAGGTTAC-3′ |
| globulin | NO: 13 | |
| forward* | | |
| Human β- | SEQ ID | 5′-CCTAAGGGTGGGAAAATAGACC-3′ |
| globulin | NO: 14 | |
| reverse* | | |
| Human β- | SEQ ID | 5′ Quasar 670? ACTGGGCATGTGGAGACAGA-BHQ2-dT 3′ |
| globulin PCR | NO: 15 | |
| probe* | | |
| MTB IS6110 | SEQ ID | 5′-CGATGTGTACTGAGATCCCCTATCCG-3′ |
| forward | NO: 16 | |
| MTB IS6110 | SEQ ID | 5′-GGCCTTTGTCACCGACGCC-3′ |
| reverse | NO: 17 | |
| MTB IS6110 | SEQ ID | 5′/56- |
| PCR probe | NO: 18 | FAM/AACGTCTTT/ZEN/CAGGTCGAGTACGCCTT/3IABkFQ/-3′ |
| MTB senX3- | SEQ ID | 5′-CAGAGCGTAGCGATGAGGTGG-3′ |
| regX3 | NO: 19 | |
| MTB senX3- | SEQ ID | 5′-GCCTCAAAGCCCTCCTTGCG-3′ |
| regX3 | NO: 20 | |
| MTB senX3- | SEQ ID | 5′-/5HEX/GAGGA + CGAGGAGT + CGC + TGGC/3BHQ_1/-3′** |
| regX3 | NO: 21 | |
| BA cotJC | SEQ ID | 5′-GGCGGCTCTTCGTTACTTAAA-3′ |
| forward | NO: 22 | |
| BA cotJC | SEQ ID | 5′-GAACTCCTCGGTCCCTATATCA-3′ |
| reverse | NO: 23 | |
| BA cotJC | SEQ ID | 5′-/d Quasar 670/TACCTGACAAAGTGATCGGGCTGC/BHQ_2/-3′ |
| PCR probe | NO: 24 | |
| Human β- | SEQ ID | 5′-ACTGGGCATGTGGAGACAGA-3′ |
| globulin PCR | NO: 25 | |
| probe* | | |
| MTB IS6110 | SEQ ID | 5′-AACGTCTTT/ZEN/CAGGTCGAGTACGCCTT-3′ |
| PCR probe | NO: 26 | |
| MTB senX3- | SEQ ID | 5′-GAGGA + CGAGGAGT + CGC + TGGC-3′ |
| regX3 | NO: 27 | |
| BA cotJC | SEQ ID | 5′-TACCTGACAAAGTGATCGGGCTGC-3′ |
| PCR probe | NO: 28 | |

*From Abravaya et al., U.S. Pat. Pub. No. 2010/0081124
**a locked nucleic acid as indicated by the +

Freeze-dried qPCR master mix was used for the DNase I experiment and for the field testing study using clinical specimens. The freeze-dried master mix composition is the same as the liquid qPCR master mix except that the amount of qPCR enzyme stabilizers were modified as follows: 2.5 mg/ml BSA, 0.03% TWEEN® 20 and 138 mM trehalose, and the bicine buffer, potassium acetate and manganese were added in the resuspension buffer. Oligonucleotides were supplied by IDT (Corvallis, Iowa) with the exception of the cotJC probe3 which was supplied by Biosearch Technologies (Petaluma, Calif.).

Amplification was performed in a 5-plex Qiagen (Hilden, Germany) Rotor-Gene Q thermocycler, with the following cycling conditions: 1. 95° C. 2:00, 2. 95° C. 0:15, 3: 60° C. 0:45, 4. repeat steps 2-3 44 times. Program was set to acquire in green, yellow and red channels, and copy number of the two MTB targets, relative to the H37Rv TMC 303 genomic DNA standard curve, was determined by using the Qiagen Rotor-Gene Q Series Software package. JMP® software (SDS Institute; Cary, N.C.) was used to calculate differences between means via one-way analysis of variance for independent samples, as well as to generate means diamonds plots. P-values of less than 0.01 were considered significantly different. Pearson correlation tests were used to generate R² values to evaluate correlations between sputum-specific factors and MTB assay inhibition.

### Example 2

### Inhibition of qPCR by Genomic DNA

After sputum analysis showed that human genomic DNA (gDNA) content correlated with inhibition of qPCR assays, studies were done to confirm that gDNA was indeed an inhibitor of qPCR by testing the effects of DNasel treatment on qPCR. A cocktail of sputum specimens was prepared by mixting 15 1-mL residual sputum specimens with approximately 7.5 g acid-washed 5 mm glass beads (Sigma; St. Louis, Mo.) for 5 minutes. Five 350 μl aliquots of this cocktail were extracted using the Dynabeads® SILANE Genomic DNA Kit (Life Technologies, Carlsbad, Calif.) as per manufacturer instructions, and the elutions were pooled. 11.5 μl of the pooled eluate was treated with 1 μl Turbo DNase I (Life Technologies, Carlsbad, Calif.), 1.5 μl Turbo DNase I buffer, and 1 μl molecular grade water in a total of 15 μl and incubated for 30 minutes at 37° C. followed by 20 minutes at 95° C. to inactivate the DNase I. The controls for this study included heat inactivated DNase I treated eluate, untreated eluate, elution buffer and elution buffer plus DNase I buffer. For the heat inactivated DNase I control, Turbo DNase I was first inactivated for 20 minutes at 95° C., and then combined with the eluted DNA and Turbo DNase I buffer as described above. PCR was performed using the MTB IS6110 assay described in Example 1. Fifteen microliters of the untreated eluate, elution buffer and elution buffer plus DNase I buffer were added directly to the PCR reaction. Five thousand copies of H37R MTB genomic DNA was amplied using the MTB IS6110 assay. Results are shown in FIGS. 1A and 1B. The standard curve is 500,000; 50,000; 5000, 500, 50 and 5 MTB H37Rv genomic DNA copies in triplicate. The equation of the line in y=−3.7X+35.5; R²=0.997. qPCR efficiency=89%.

A box and whisker plot of the data is provided in FIG. 1B. Center lines show the medians; box limits indicate the 25th and 75th percentiles as determined by R software; whiskers extend 1.5 times the interquartile range from the 25th and 75th percentiles, outliers are represented by dots; crosses represent sample means; data points are plotted as open circles. Sample points per group are represented on the x-axis, as n=6, 3, 3, 6. Heat-killed DNase I data not shown because 4 of the 6 replicates failed to amplify.

Relief of inhibition was observed in the specimens that contained active DNase I (FIG. 1B). The Tris buffer control had an average Cq of 22.0±0.1, and the untreated eluate from sputum extraction had an average Cq of 26.0±0.3. This four Cq delay would lead to more than a 10-fold under-quantification of the MTB bacterial load. Treatment of eluate with DNase I allowed a mean recovery of 5.4 Cq. DNase I activity was required for this relief of inhibition because heat-inactivated DNase I did not improve amplification (data not shown). In fact, 4 of the 6 replicates incubated with inactivated DNase I failed to amplify while the remaining two samples had Cqs of 24.6 and 28.5, similar to the untreated eluate. Eluates from 3 additional sputum extractions gave analogous results with DNase I treatment (data not shown) indicating that human genomic DNA in sputum is a frequent and potent qPCR inhibitor.

### Example 3

### Sputum Processing and Nucleic Acid Preparation

To demonstrate the advantages imparted by removing gDNA from samples three different sputum thinning and decontamination methods were compared using 16 individual sputum specimens (FIG. 3a): N-acetyl-L-cysteine-sodium hydroxide (NALC), guanidinium hydrochloride (GuHCl) which is the first step of the Dynabeads® SILANE Genomic DNA Kit (Life Technologies, Carlsbad, Calif.), and sodium dodecyl sulfate with proteinase K (SDS/PK). NALC and GuHCl thinning was used to prepare bulk DNA while SDS/PK treatment was used prior to sequence-specific capture and preparation of DNA for PCR amplification.

### Bulk DNA Preparation

To thin sputum specimens by NALC, fresh NALC reagent was prepared by adding equal volumes of 4% NaOH and 2.9% sodium citrate with 0.5 g NALC per 100 ml NaOH/citrate solution (Kent, 1985, Public Health Mycobacteriology: a guide for the level III laboratory, G. P. Kubica, Editor, U.S. Dept. of Health and Human Services, Public Health Service, Centers for Disease Control: Atlanda, Ga.). To each sputum specimen, an equal volume of the NALC reagent was added and mixed by vortexing, then incubated for 15 minutes at room temperature with intermittent shaking. The samples were neutralized by the addition of phosphate buffer (pH 6.8) at a ratio of 1:15 (sample:phosphate buffer) and mixed by vortexing. The pellet was collected via centrifugation for 15 minutes at 3000×g, resuspended with 350 μl phosphate buffer and immediately processed using the Dynabeads® SILANE Genomic DNA Kit according to the manufacturer's instructions.

To thin sputum specimens by GuHCL, reagents included with the Dynabeads® SILANE Genomic DNA Kit were used and the thinned sputa were immediately processed according to the manufacturer's instructions.

### Sequence-Specific DNA Preparation

Sputum thinning using SDS/PK was performed by treating sputum with SDS/PK followed by sequence specific capture. Treatment with SDS/PK included use of a tube containing dried thinning reagent. Two tubes per reaction were prepared with dried reagents: one for sputum thinning and one for specific capture probe (oligonucleotide) binding. To make the tube containing dried thinning reagent, a sputum thinning buffer was prepared by combining 50 μl 20% SDS, 30 μl 1 M Tris, pH 8.0; 20 μl 0.5 M EDTA, pH 8.0 per reaction for a final concentration in 1 ml of 1% SDS, 30 mM Tris pH 8.0 and 10 mM EDTA, pH 8.0. 100 μl of this SDS solution was pipetted onto the side of sterile 1.5 ml conical-bottom tubes. The tubes were kept in a horizontal position and dried in a 55° C. oven overnight.

Sequence-specific capture involved preparing a tube of dried oligonucleotide binding buffer and a biotin-labeled oligonucleotide cocktail mix.

To make tubes of dried oligonucleotide binding buffer, an oligonucleotide binding buffer was prepared by combining 50 μl 5 M NaCl, 10 μl 1 M Tris, pH 8.0, 2 μl 0.5 M EDTA, pH 8.0 and 0.5 μl 10% TWEEN® 20 per reaction for a final concentration of 250 mM NaCl, 10 mM Tris, pH 8.0, 1 mM EDTA and 0.005% TWEEN® 20. 62.5 μl oligo binding buffer was pipetted onto the side of a 1.5 ml sterile conical tube and kept in a horizontal position while dried in a 55° C. oven overnight. The tubes were capped and stored at room temperature in aluminum moisture-barrier pouches (Ted Pella; Redding, Calif.), with silica gel desiccant (McMaster Carr; Elmhurst, Ill.) to maintain dryness and a humidity indicator card (Static Control Components; San Diego, Calif.) to monitor moisture.

The cocktail of biotin-labeled oligonucleotides (see Table 1) was prepared in advance wherein 5 μl of the cocktail contained a total of 5 or 5.5 pmols of probe: for the singleplex IS6110 assay, 2.5 pmols of IS6110 capture probes 1 and 2 were used, and for the multiplexed assay, 1.25 pmols each of IS6110 capture probes 1 and 2, 1.25 pmols each of senX3-regX3 capture probes 3 and 4, and 0.25 pmols each of cotJC capture probe 1 and capture probe 2 were used. Capture probes were obtained from Integrated DNA Technologies (IDT; Coralville, Iowa), were diluted in 10 mM Tris pH 8, and stored at −20° C. until time of use. All capture oligonucleotides were labeled at the 5′ end with a biotin moiety, contained a spacer of 5 adenine residues upstream of the specific sequences described above (Table 1), and were HPLC-purified. Next, to initiate sputum thinning, 950 μl sputum, 50 μl of Proteinase K solution containing 30 U Proteinase K, and 1 μl 1 M CaCl₂ were added to a 1.5 ml tube containing dried thinning reagent. The tubes were then heated to 55° C. for 8 minutes with mixing at 1000 rpm (Benchmark Scientific, Inc; South Plainfield, N.J.). The temperature was then ramped to 95° C., and the specimens were incubated for 10 minutes with 1000 rpm mixing.

Samples were removed from the thermal mixer, spun briefly, and the entire volume was transferred to the tube containing dried oligonucleotide binding buffer. Five microliters of biotin-labeled oligonucleotide cocktail mix (5 pmols in total) was added, and the samples were incubated at 60° C. and 1000 rpm for 20 minutes. Twenty microliters of pre-washed Dynal Streptavidin M-270 paramagnetic particles (PMPs) (Thermo Fisher Scientific; Waltham, Mass.) were added and samples were incubated for 10 minutes at room temperature with end-over-end rotation. The liquid was collected to the bottom of the tube by a short centrifugation step and the PMPs were pelleted on a magnetic rack (DynaMag2™) for 2 minutes. Supernatants were aspirated, the PMP pellet was resuspended in 1 ml wash buffer (10 mM Tris, pH 8.0 & 0.01% Tween® 20), and the entire volume was transferred to a new 1.5 ml tube. Again, PMPs were collected on a magnetic rack, followed by supernatant aspiration and resuspension of PMPs in 1 mL wash buffer. Following this second wash, PMPs were collected on the magnetic rack and the supernatant was aspirated, taking care to remove all remaining wash buffer volume. The pellet was carefully resuspended in 10 μl elution buffer (10 mM Tris, pH 8.0, 0.01% Tween® 20, and 10% glycerol) and heated at 75° C. with 1500 rpm mixing for 3 minutes. It is important that PMPs are resuspended in the elution buffer during the heated elution step. The liquid was collected at the bottom of the tube by a brief spin, and the samples were placed on the magnetic rack to collect PMPs. The 10 μl elution was carefully transferred to clean tube ready for qPCR.

### Quantification of Genomic DNA

For the sputum samples processed by the NALC, GuHCl or SDS/PK methods as described above, the human DNA and MTB DNA was measured. Human DNA was quantified using a standard curve of 100 to 1,000,000 copies of β-globulin plasmid DNA. MTB yield was quantified via a standard curve of 100 to 100,000 copies of IS6110 Mycobacterium bovis BCG genomic DNA. Samples were run at 10 μl eluate, 10 μl of 10⁻¹ dilution and 10 μl of 10⁻² dilution in duplicate to insure that the Cq would be on the standard curve and to use the dilutions to determine if amplification was inhibited. For the GuHCl samples, we quantified the β-globulin gene using the 10⁻² dilution and determined the inhibition by comparing the Cqs of the 10⁻¹ and 10⁻² dilutions. For the NALC treated samples, we quantified the β-globulin gene using the 10⁻¹ dilutions and determined inhibition by comparing the Cqs of the neat samples vs. the 10⁻¹ dilutions. For the SDS/PK treated samples, we quantified the β-globulin gene using the neat eluates and determined inhibition by comparing the Cqs of the neat samples vs. the 10⁻¹ dilutions. For IS6110, all 3 conditions, GuHCl, NALC and SDS/PK were quantified using the 10⁻¹ dilutions, and inhibition was identified by comparing the Cqs of the neat eluates vs. the 10⁻¹ dilutions.

MTB DNA yield measured in IS6110 copies (FIG. 3B), human gDNA yield measured in β-globulin copies (FIG. 3C), and qPCR inhibition was compared between the three strategies. In the box and whisker plots shown in FIGS. 3B and 3C, the center lines show the medians; box limits indicate the 25^{th} and 75^{th} percentiles as determined by R software; whiskers extend 1.5 times the interquartile range from the 25^{th} and 75^{th} percentiles with outliers represented by dots; crosses represent sample means; bars indicate 95% confidence intervals of the means; data points are plotted as open circles. Each group is represented by n=16. ** denotes difference in mean between the different conditions that are significant p<0.01.

By using the SDS/PK thinning technique with MTB specific capture, we were able to reduce the average amount of human genomic DNA co-extracted 2.5 to 3.7 logs. Mean β-globulin copy number co-extracted from 350 μl of sputum was 6.2±0.8 log for NALC treatment, 7.4±1.2 log for GuHCl treatment, and 3.7±0.8 log for SDS/PK treatment.

### Example 4

### qPCR of Processed Sputum Samples

Sixteen MTB-negative sputum specimens with sufficient volume (≧1.8 mls) were selected for a study to compare qPCR using samples prepared by the various methods. MTB-spiked sputum specimen were thinned in one of 3 ways: 1) NALC, 2) GuHCl, or 3) SDS/PK, and 350 μl of the thinned sputa were extracted by either Dynabeads® SILANE Genomic DNA Kit (NALC & GuHCl) or sequence specific capture (SDS/PK) (FIG. 3A). Human gDNA yield measured in β-globulin copies (FIG. 3B), MTB DNA yield measured in IS6110 copies (FIG. 3C), and qPCR inhibition was compared between the three strategies. By using the SDS/PK thinning technique with MTB specific capture, we were able to reduce the average amount of human genomic DNA co-extracted 2.5 to 3.7 logs. Mean β-globulin copy number co-extracted from 350 μl of sputum was 6.2±0.7 log for NALC treatment, 7.4±1.4 log for GuHCl treatment, and 3.7±0.7 log for SDS/PK treatment. None of the NALC-treated or SDS/PK-treated samples were qPCR inhibited. However, 75% (12/16) of the GuHCl treated specimens exhibited qPCR inhibition with the amount of β-globulin co-extracted corresponding to qPCR inhibition (FIG. 4). The mean β-globulin log copy yield of GuHCl treated samples for IR0 (5.9±1.6) was significantly different (p<0.01) from IR1 (7.8±0.5) and IR2 (8.1±0.4), but the mean yield of IR1 and IR2 were not significantly different. The MTB yield was measured using 10¹ dilutions of the eluate to sidestep the effect of qPCR inhibition. There were no significant differences between mean MTB yield of the GuHCl treated specimens (4.7±1.0 log copies), the NALC treated specimens (4.1±0.6 log copies), or the SDS/PK treated specimens (4.1±0.8 log copies).

At least 1 ml of sputum must be extracted and amplified in order to achieve equivalent sensitivity to culture (10 cfu/ml) {van Zyl-Smit, 2011 #80}. Because we added only 10% of the eluate from a 350 μl specimen, we used roughly 30-fold less specimen than that. If we multiply the average genomic DNA yield by a factor of 30, the average yield of β-globulin copies would increase to 7.7 log for NALC, 8.9 log for GuHCl, and 5.2 log for SDS/PK thinning with specific capture extraction. The amount of co-extracted β-globulin DNA in the NALC treated specimens would then be equivalent to the average amount of DNA found in the inhibited GuHCl specimens, suggesting that 1 ml NALC-treated samples would also likely be inhibited. However, the SDS/PK treated specimens average gDNA yield is still much below that level. Due to the fact that specific capture based DNA extraction resulted in equivalent IS6110 copies (FIG. 3B), but significantly lower human gDNA (FIG. 3C), it was selected as the preferred nucleic acid extraction strategy for the MTB diagnostic assay.

### Example 5

### Multiplexed qPCR Assay

To test the sequence-specific capture sample preparation method with genuine TB-suspect specimens, an assay was developed to detect all MTB strains by multiplexing the IS6110 assay with primers and probes specific to an intergenic region in the two-component regulatory operon senX3-regX3. An amplicon of the Bacillus atrophaeus cotJC gene that encodes a spore coat composition polypeptide (Seyler et al, 1997, Mol Microbiol, 25:955-966) was also included to monitor the test for extraction, qPCR inhibitors, and amplification efficiency. The B. atrophaeus spores were added to the sputum prior to sample processing and were thinned and lysed along with the MTB. Capture probes were designed for each specific target (Table 1 above), tested in isolation and then in combination for optimal specific MTB DNA extraction (FIG. 5). Contrived sputum specimens of 5000 cfu of MTB H37Ra and 500 B. atrophaeus spores were in triplicate extracted using the specific capture MTB DNA extraction method, and the multiplexed PCR assay was performed to confirm capture of multiple targets and assay performance. Horizontal black line is threshold. Solid black line is IS6110. Dashed black line is senX3-regX3. Dark gray line is cotJC, and light gray line is no template control (NTC). The IS6110 amplicon had an average Cq of 24.8±0.4, the senX3-regX3 had an average Cq of 28.7±0.5 and the cotJC amplicon had an average Cq of 32.1±0.6 (FIG. 5). The data show that all 3 targets are efficiently captured and amplified from sputum.

To estimate the limit of detection, 1 ml contrived sputum specimens containing 100, 50 or 20 cfu/ml, and six replicates of each concentration were assayed. All 6 of the 50 and 100 cfu/ml specimens were detected by both the 156110 and senX3-regX3 assays. Five of the 20 cfu/ml specimens were detected by the 156110 assay, and none of these samples were detected by the senX3-regX3 assay. Therefore, the LOD of the combined assay was estimated as 20 cfu/ml for the combined assay. One of the 20 cfu/ml samples was excluded from analysis because the Cqs in both assays were anomalously low. MTB has a tendency to clump, and despite the fact that the bacterial preparation was sonicated to break up clumps before serially diluting the samples for contriving the sputum specimens, we suspect that this sample had a clump of bacteria. The assay was also tested with 6 different Mycobacterium species for potential cross reactivity. M. gordonae, M. intracellulare, M. terrae, M. malmoense, M. celatum, and M. abscessus were added to qPCR reactions at 50 000 copies/PCR reaction, and no amplification was observed.

### Example 6

### Laboratory Validation Study of Sputum Processing and Work Flow

To determine if the SDS/PK sputum pre-treatment combined with sequence-specific capture is effective in processing clinical specimens, a small-scale laboratory validation study was performed at the routine TB diagnostic laboratory at Groote Schuur Hospital, Cape Town, South Africa. A convenient sample of 60 blinded routinely collected specimens that had ≧1 ml of sputum left over after GeneXpert® (Cepheid, Sunnyvale, Calif.) testing were tested using the multiplexed MTB assay protocol described here; 27 MTB/RIF positive specimens and 33 negative specimens were analyzed. One of the Xpert negative specimens was invalid (no Cq in cotJC test). All 59 distinct sputum samples thinned to a pipetable liquid in the sputum thinning step. Of the 27 positive specimens, three samples were negative in senX3-regX3 assay and one of these same samples was also negative in the IS6110 assay yielding a sensitivity of 88.9% (24/27) and 96.3% (26/27), respectively (Table 3). One of the Xpert® MTB/RIF negative specimens failed to amplify the cotJC gene and was called invalid. The sample that was a false negative, having failed in both the IS6110 and senX3-regX3 assays, was reported as a very low positive in the Xpert® MTB/RIF assay. The 32 negative specimens with valid test results were also negative in the IS6110 and senX3-regX3 assays, yielding 100% specificity in both assays.

TABLE 3

| | Positive | Negative | Sensitivity or Specificity (CI) |
|---|---|---|---|
| IS6110 positive | 26 | 0 | 96.3% (79.1-99.8) |
| IS6110 negative | 1 | 32 | 100% (86.7-100) |
| Totals | 27 | 32 | 59 |
| senX positive | 24 | 0 | 88.9% (69.7-99.1) |
| senX negative | 3 | 32 | 100% |
| Totals | 27 | 32 | 59 |

Another study was done using clinical sputum samples collected from 94 adults showing symptoms of pulmonary TB at participating clinics and that were donated to the FIND Tuberculosis Specimen Bank. Although these repository samples are linked to clinical and microbiological information available from FIND, the specimens were de-identified, and TB status (as reflected by smear microscopy, culture, chest x-ray, clinical symptoms and MTB/RIF Gene Xpert Assay®) was blinded from sample processors.

### Sputum Sample Processing and MTB Specific Capture DNA Extraction

Tubes containing dried reagents for sputum thinning and capture probe hybridization (binding) were prepared prior to DNA extractions as follows. Thinning buffer reagents (50 μl 20% SDS, catalog number 05030-1L-F, Sigma Aldrich, St. Louis, Mo.), 30 μl 1M Tris pH8 (cat. no 15568-025, Invitrogen, Carlsbad, Calif.), 20 μl 0.5M EDTA pH 8 (cat. no AM9260G, Ambion, Carlsbad, Calif.) were combined, and 100 μl of this buffer was pipetted on the inside wall of a sterile 1.5 ml screw-top tube (cat. no 89004-290, VWR, Radnor, Pa.) and allowed to air dry overnight in a 55° C. oven. Binding buffer reagents (50 μl 5M NaCl (S5150, Sigma), 10 uL 1M Tris pH 8, 2 μl 0.5M EDTA pH 8, and 0.5 μl 10% Tween-20 (cat. no 28320, Thermo Scientific, Waltham, Mass.) per tube) were similarly combined and dried. After drying, the tubes were capped and stored at room temperature in aluminum moisture-barrier pouches (Barrier Foil Ziplock Pouch, 6.25×7.75×2.5″, cat. no 139-312, Ted Pella, Redding, Calif.), with silica gel desiccant (cat. no 2189K46, McMaster Carr, Elmhurst, Ill.) to maintain dryness and humidity indicator cards (cat. no 51015HIC125, Static Control Components, Sanford, N.C.) to monitor for moisture.

A cocktail of biotin-labeled capture probes was prepared in advance such that 5 μl contained: 1.25 pmols IS6110 capture probe 1, 5′-CGAACGGCTGATGACCAAACTC-3′ (SEQ ID NO:7), 1.25 pmols IS6110 capture probe 2, 5′-GGAGGTGGCCATCGTGGAAG-3′ (SEQ ID NO:8), 2.5 pmols SenX-RegX capture probe 5, 5′-CAGAGCGTAGCGATGAGGTGGG-3′ (SEQ ID NO:29), 0.25 pmols BATR capture probe 1 5′-CAAATACTTAATCGAGCAATATGGCGGCG-3′ (SEQ ID NO:30), 0.25 pmols BATR capture probe 2, 5′-GCTTATACACCATCGTCGCAATCATCTCC-3′ (SEQ ID NO:31) was added to each tube. All capture probes were labeled at the 5′ end with a biotin moiety, have a spacer of 5 adenine residues prior to the specific sequences described above, and were HPLC-purified. Probes were obtained from Integrated DNA Technologies (IDT, Coralville, Iowa), were diluted in 10 mM Tris pH8, and were stored at −20° C. until time of use. Immediately prior to the DNA extraction, Tris-Tween-Glycerol solution (10 mM Tris pH 8, 0.01% Tween-20, 10% glycerol (cat. no 16374, Affymetrix, Santa Clara, Calif.)) was prepared. A stock of H37Rv TMC 303 genomic DNA (cat. no 359220-2, ATCC) was diluted to 104 copies/μl in this Tris-Tween-Glycerol solution. This genomic DNA was kept on ice and was used for positive extraction controls. Proteinase K (cat. no 25530-031, Life Technologies, Carlsbad, Calif.) was diluted in a solution of 50% glycerol, 10 mM Tris pH 8 to 30 mg/ml. 50 μl of this Proteinase K solution was combined with 1 μl of 1 M calcium chloride (cat. no 21115, Sigma Aldrich) per sample and was kept on ice until use. Bacillis atrophaeus spore stock (spore suspension, 104 CFU/μl, from ATCC No. 9372, Mesa Biological Indicators, Omaha, Nebr.) was strongly vortexed and was diluted to 102 CFU/μl in 40% ethanol and was then kept on ice.

All clinical sputum sample processing steps prior to heat-killing of bacteria were performed in a biological safety cabinet. Because the sputum sample volume was variable, sputum samples were thawed and sample input was normalized to a total volume of 950 uL. In 29 of the 94 cases, ultrapure molecular-grade water (range: 50-750 μl) was added in order to equalize volume across the panel.

Two positive and two negative extraction controls were prepared by adding 950 μl 10 mM Tris pH 8 to the dried thinning buffer tubes. Sputum samples (at a total volume of 950 μl) were then transferred into pre-labeled dried thinning buffer tubes. Fifty thousand copies (5 μl) of the prepared H37Rv genomic DNA stock were added to the positive extraction control tubes only. One thousand CFU of the prepared B. atrophaeus stock was then added to all samples. Finally, 51 μl of the prepared Proteinase K/calcium chloride solution was added to each sample. All samples were vortexed and quickly spun prior to loading into a Benchmark Multi-Therm heater-shaker (cat. no H5000-HC, Benchmark Scientific, Edison, N.J.) pre-set to 55° C. and programmed to hold at temperature with 1500 rpm shaking for 8 minutes. After 3 minutes, all samples were vortexed briefly to ensure complete mixing, and put back in the heater-shaker for the remaining time programmed. Immediately following this step, the heater-shaker was set to 100° C. with 1500 rpm shaking. Samples remained in the heater-shaker as the temperature ramped up, and were held at 100° C. for 10 minutes.

During this incubation, dried binding buffer tubes were labeled with sample ID information, and two further sets of clean 1.5 mL screw-top tubes were prepared and labeled. Dynabeads M-270 Streptavidin paramagnetic particles (cat. no 65305, Invitrogen, Carlsbad, Calif.) were vortexed for 30 seconds, aliquoted into a 1.5 mL screw-top tube (accounting for 20 uL per sample processed) and collected on a magnetic stand. The storage buffer was removed, and the paramagnetic particles (PMPs) were washed two times in 1 mL Tris-Tween buffer (10 mM Tris pH 8, 0.01% Tween-20). Finally, they were resuspended in their initial volume in Tris-Tween buffer and set aside at room temperature.

Following the 100° C. step, all samples were given a quick vortex and spin, and the heater-shaker was re-set to 60° C. The entire volume of thinned sample was transferred to prepared binding buffer tubes containing binding buffer reagents. 5 μl of capture probe cocktail prepared as described above was added to each sample. Samples were vortexed, given a quick spin, and were then loaded into the heater-shaker set to hold at 60° C. for 20 minutes.

Samples were removed and briefly spun, and 20 μl of PMPs were added to each sample, vortexing the PMP stock periodically to ensure PMPs did not settle. Samples were vortexed, and placed on an end-over-end roller at room temperature for 10 minutes. Samples were then loaded onto a magnetic stand where PMPs were collected, and the supernatant was discarded into a waste container. PMPs were washed in 1 ml Tris-Tween buffer and transferred to a clean pre-labeled tube. PMPs were collected and washed again in 1 ml Tris-Tween buffer, for a total of two washes. All remaining wash solution was carefully removed following the second wash. Finally 11.75 μl of Tris-Tween-Glycerol buffer was added to each test sample, and 20 μl was added to each positive control sample. Samples were vortexed well and given a very light spin to collect all droplets, but not pellet the PMPs. Samples were placed in the heater-shaker pre-set to 75° C., and were incubated with 1500 rpm for 3 m. Samples were vortexed and placed on the magnetic stand. Eluted DNA was transferred to a clean tube and was placed on ice until PCR.

### PCR Amplification

A standard curve of H37Rv TMC 303 genomic DNA was serially diluted 1:10 in Tris-Tween-Glycerol (105-10-1 copies/μl) and was kept on ice. Dilutions (1:10) of the positive extraction controls were prepared in Tris-Tween-Glycerol also and were kept on ice. PCR master mix for a 25 μl total reaction volume (15 μl master mix and 10 μl of eluted DNA) was made as follows (concentrations listed are final in 25 μl: 0.2 mg/ml bovine serum albumen (cat. no AM2616, Ambion), 0.2 mg/ml Tween-20, 150 mM Trehalose (cat. no T9531, Sigma), 10% glycerol, 62.5 mM bicine pH 8 (cat. no 12091, Affymetrix), 135 mM potassium acetate pH7.5 (cat. no 20602, Affymetrix), 2.0 mM magnesium chloride (cat. no M1028, Sigma), 0.325 mM each dNTP (cat. no 10297-117, Invitrogen), 0.375 U RMS Z05 DNA polymerase (cat. no L1188, Roche Diagnostics, Risch-Rotkreuz, Switzerland), and sequence-specific oligonucleotides. Oligonucleotides supplied by IDT with their final concentrations are as follows: 200 nM IS6110 F8 5′-CGATGTGTACTGAGA TCCCCTAT-3′ (SEQ ID NO:33), 200 nM IS6110 R11 5′-CTTTGTCACCGACGCC-3′ (SEQ ID NO:34), 300 nM IS6110 probe 5′-FAM/AACGTCTTTCAGGTCGAGTACGCCTT-3′ (SEQ ID NO:35), 300 nM SenX-RegXF5 5′-AGAGCGTAGCGATGAGGT-3′ (SEQ ID NO:36), 200 nM SenX-RegXR3 5′ CTCAAAGCCCTCCTTGCG-3′ (SEQ ID NO:32), 200 nM SenX-RegX probe 10 5′-HEX/CTCAAAGCCCTCCTTGCG-3′ (SEQ ID NO:37), 100 nM BATR570 F2 5′GGCGGCTCTTCGTTACTTAAA-3′ (SEQ ID NO:38), 100 nM BATR570 R2 5′-GAACTCCTCGGTCCCTATATCA-3′ (SEQ ID NO:23). The probe for the BATR assay was obtained from Biosearch Technologies (Petaluma, Calif.): 100 nM BATR570 probe3 5′-Quasar670/TACCTGACAAAGTGATCGGGCTGC-3′ (SEQ ID NO:24).

Master mix was loaded into PCR tubes in a cold block. Ten microliters of Tris-Tween-Glycerol buffer was added to the NTC tubes, and tubes were then capped. No DNA extraction controls were then loaded, followed by a B. atrophaeus gBlock DNA positive control (1000 copies). Standard curve DNA was then added to appropriate tubes (final range: 106-1 copies/PCR reaction). Finally, eluted DNA and 10× dilutions were added to appropriate tubes (singly or in duplicate, respectively).

All tubes were capped and were run on a 5-plex Qiagen (Hilden, Germany) Rotor-Gene Q thermocycler machine, with the following cycling conditions: 1. 95° C. 2:00, 2. 95° C. 0:15, 3: 60° C. 0:45, 4. repeat steps 2-3 44 times. Program was set to acquire in green, yellow and red channels, and copy number for all three targets, relative to the H37Rv TMC 303 genomic DNA standard curve, was determined by using the Qiagen Rotor-Gene Q Series Software package. PCR products were frozen at −20° C. The results are provided below in Table 4.

TABLE 4

| Category* | Reference | Test | % Sensitivity | 95% CI |
|---|---|---|---|---|
| SN/CP | 30 | 29 | 97 | 80.9-99.8% |
| SCA/CP | 20 | 19 | 95 | 73.1-99.7% |
| SP/CP | 20 | 20 | 100 | 80.0-100% |
| SCA & SP/CP | 40 | 39 | 98 | 85.3-9.8% |
| Total CP | 70 | 68 | 97 | 89.1-99.5% |
| CXR/CN | 4 | 4 | 25 | |
| CN | 20 | 18^{#} | 90 | 66.9-98.2 |

*SN = smear negative; SP = smear positive; CN = culture negative; CP = culture positive; CXR = x-ray positive; SCA = positive by clinical symptoms

An extremely high overall assay sensitivity was confirmed with regard to culture-positive specimens (97%, N=70) and specificity with regard to true negative specimens (100%, N=18). This was true even in the context of smear-negative, culture-positive samples (within-group sensitivity: 97%, N=30). Impressively, 94% of smear-negative, culture-positive sputa with matched Xpert® MTB/RIF Assay data available tested positive in the CIGHT MTB Screening Assay. This population of sputum has historically been the most challenging to diagnose, and in this study, it was determined that more than half of the samples we tested in this group had less than 100 genomic copies of senX3-regX3 as normalized to MTB H37Rv genomic DNA. This emphasizes the requirement of a highly sensitive test that has minimal qPCR inhibition in order to efficiently detect MTB in smear negative specimens.

### Example 7

### Capture Probe Hybridization in the Presence of Sodium and Magnesium Ions

Further experimentation with the sequence-specific capture technique described above was done to increase the speed and efficiency of hybridization of the biotinylated capture probe to the DNA prepared by the SDS/PK processing method. For example, an increased speed of nucleic acid probe/target hybridization with a combination of sodium and magnesium cations was initially observed during extraction of the MTB genome (H37Rv) from 10 mM Tris (pH=8) −0.01% TWEEN® 20 buffer (TT buffer) or from synthetic sputum (10 mg/mL mucin, 1 mg/mL salmon DNA, 3.6 mg/mL Phosphatidylcholine (PC), 33 mg/mL BSA, and 114 mM NaCl). To further characterize this effect, DNA preparation from sputum was performed in the presence of NaCl and/or MgCl₂ as follows.

950 μl of artificial sputum spiked with 50,000 copies of H37Rv gDNA was added to a 1.5 mL tube containing dried thinning buffer. To make dried thinning buffer, liquid thinning buffer consisting of Tris (pH=8) and SDS was pipetted onto the side of a 1.5 ml tube and dried in a 55° C. oven overnight as described in Example 3. The amounts of Tris (pH=8) and SDS were such that the concentrations of each become 30 mM and 1%, respectively, when resuspended in 1 ml. Next, 50 μl of 30 mg/ml LifeTech fungal Proteinase K with 20 mM CaCl₂ was added to the sample. The sample tube was mixed at 55° C. and 1500 rpm for 8 minutes on a Benchmark Multi-Therm to allow for protein digestion via proteinase K and SDS. The sample was then mixed at 100° C. and 1500 rpm for 10 minutes to denature the double stranded DNA. After cooling, the sample was transferred to a 1.5 ml tube containing dried binding buffer.

To make dried binding buffer, liquid binding buffer consisting of Tris (pH=8), TWEEN® 20 and differing amounts of sodium chloride and magnesium chloride, was pipetted onto the side of a 1.5 ml tube and dried in a 55° C. oven overnight. The amounts of the buffer components are such that the concentrations of each becomes 10 mM Tris, 0.005% TWEEN® 20, and: 500 mM NaCl, 600 mM NaCl, 700 mM NaCl, or 500 mM NaCl/50 mM MgCl₂. Next, oligonucleotide probes (all with a 5′ biotin and 5-adenine linker modification; see Table 1) were added to the sample. Three different capture probe sequences were used. The target of two of the probes is IS6110, a transposable element that appears sixteen times in the MTB H37Rv genome. The sequences of the IS6110 capture probes are 5′-CGAACGGCTGATGACCAAACTC-3′ (SEQ ID NO:7) and 5′-GGAGGTGGCCATCGTGGAAG-3′ (SEQ ID NO:8). The target of the third probe is senX3-regX3, a two component regulatory mechanism system in the MTB genome. The sequence of the senX3-regX3 capture probe is 5′-CAGAGCGTAGCGATGAGGTGGG-3′ (SEQ ID NO:29). 1.25 pmol of each IS6110 probe and 2.5 pmol of the senX3-regX3 probe were added to the sample for a total of 5 pmol of capture probe. Then sample was incubated at 60° C. and mixed at 1500 rpm for varying amounts of during which capture probe/target hybridization occurs. After this incubation, streptavidin-coated m270 paramagnetic particles (PMPs) by Invitrogen (Life Technologies) were washed two times with TT buffer, and 20 μl were added to the sample. The solution was mixed by inversion for ten minutes at room temperature to allow the streptavidin-biotin binding. The sample was then placed on a magnetic stand to collect the PMPs. The supernatant was discarded and the PMPs underwent two washes/collections with TT buffer. After the final wash was removed, the PMPs were resuspended in 20 μl of elution buffer (10% glycerol, 0.01% TWEEN® 20, 10 mM Tris pH=8). The sample was eluted at 75° C. for 3 minutes at 1500 rpm. The PMPs were collected, the eluate was transferred to a clean tube, and 10 μl of the eluate was amplified via qPCR using standard methods (see, e.g., Example 1). All conditions were run in triplicate. For each condition, Table 4 below reports average Cq, average percent capture, average ΔCq from 500 mM NaCl with a 20 minute hybridization incubation, and average separation from its ten-fold dilution. All data are based on gDNA capture with IS6110 probes. The condition of 500 mM NaCl with 20 minute incubation is a benchmark for comparison of the other salt additions and incubation times.

TABLE 5

| | | | | | | | Cycle |
|---|---|---|---|---|---|---|---|
| | | | | | | | Separation |
| NaCl | MgCl₂ | Incubation | | Std. | | Percent | from |
| (mM) | (mM) | time | Cq | Dev. | ΔCq* | Capture | Dilution |
| 500 | 0 | 20 | 20.66 | 0.45 | — | 22% | 3.25 |
| 500 | 0 | 5 | 21.36 | 0.13 | 0.70 | 14% | 3.34 |
| 600 | 0 | 20 | 20.40 | 0.09 | −0.26 | 26% | 3.30 |
| 600 | 0 | 5 | 20.96 | 0.11 | 0.30 | 18% | 3.51 |
| 700 | 0 | 20 | 20.30 | 0.08 | −0.36 | 28% | 3.50 |
| 700 | 0 | 5 | 21.07 | 0.19 | 0.41 | 17% | 3.50 |
| 500 | 50 | 20 | 19.61 | 0.12 | −1.05 | 44% | 3.45 |
| 500 | 50 | 5 | 20.23 | 0.18 | −0.43 | 29% | 3.36 |

*relative to Cq with 500 mM NaCl, 0 MgCl₂, 20 min incubation

More DNA was captured in the 20 minute incubation times in all salt conditions. However, 500 mM NaCl with 50 mM MgCl₂ and a 5-minute hybridization incubation isolated more target DNA than the 20 minute hybridization incubation with 500 mM NaCl. Thus supports the assertion that magnesium cations in combination with sodium cations increase the rate of probe/target hybridization compared to sodium cations alone. Data from the experiment support that use of sodium and magnesium cations together in the incubation solution to produce faster probe/target hybridization than sodium ions alone. This study was designed to demonstrate that it was the specific addition of magnesium and not the increase in ionic strength that caused the faster hybridization.

### Example 8

### Mycobacterium tuberculosis Molecular Screening Test with Sensitivity Approaching Culture

Experiments were conducted during development of embodiments herein demonstrate a MTB testing protocol useful for inclusion in either an integrated point-of-care platform or a high throughput automated central laboratory system. The test combines DNA sequence specific sample prep to reduce the co-extraction of qPCR inhibitors with the amplification of two MTB specific loci (IS6110 and senX3-regX3) to increase test sensitivity and minimize the likelihood of false negatives. The analytical sensitivity of the XtracTB Assay was 5 genomic copies/ml of sputum rivaling that of culture. 142 valid test results yield clinical sensitivity of 94.9% (95% CI: 90.1-99.9) and specificity of 100% (95% CI: 90.0-100.0).

### Materials and Methods

### Bacterial Strains, Genomic and Plasmid DNA and Sample Sources

Mycobacterium tuberculosis H37Ra was acquired from the American Type Culture Collection (ATCC; Manassas, Va.), and sonicated to break up cell clumps prior to contriving sputum specimens as described by Helb, et al. 15. Bacillus atrophaeus spores (MesaLabs; Lakewood, Colo., USA) were used as the process control. Residual sputa used in precision and LOD studies were obtained from TriCore Reference Laboratories (Santa Fe, N. Mex.). The recipe for synthetic sputum was adapted from Du et al. 29 and consisted of 10 mg/ml porcine mucin (Sigma Aldrich, St Louis Mo., USA), 1 mg/ml salmon sperm DNA (Sigma Aldrich), 3.6 mg/ml phosphatidylcholine (Sigma Aldrich), 33 mg/ml bovine serum albumin (Sigma Aldrich), and 114 mM NaCl (Sigma Aldrich).

This report includes two studies that used clinical sputum samples collected from 94 and 150 adults showing symptoms of pulmonary TB at participating clinics that were donated to the Foundation for Innovative New Diagnostics (FIND) Tuberculosis Specimen Bank. Although these repository samples are linked to clinical and microbiological information available from FIND, the specimens were de-identified, and TB status (as reflected by concentrated smear microscopy, culture, chest x-ray, clinical symptoms and MTB/RIF Gene Xpert Assay) was blinded from sample processors.

All studies presented here were performed in a research laboratory designed for real-time PCR assays so that DNA extraction, PCR master mix preparation, and DNA amplification are performed in separate rooms. Negative controls were performed with every study to monitor for potential qPCR contamination.

### Cloning of Lambda Phage Process Control

Synthetic DNA fragments (gBlocks) containing a portion of the cotJC gene of Bacillus atrophaeus were ordered from Integrated DNA Technologies (IDT, Coralville, Iowa, USA), and were amplified using primers with EcoRI restriction sites at the 5′ end of each primer (F primer: 5′-TTT TTG AAT TCT CAA TCA GCC ATT GGT AGG TC-3′ (SEQ ID NO:39); R primer: 5′-TTT TTG AAT TCA GCT GCA ATA TCC TGT AAA GGT C-3′ (SEQ ID NO:40)) and ligated into Lambda Zap II vector predigested with EcoRI (Agilent, Santa Clara, Calif., USA) using T4 DNA ligase (New England Biolabs, Ipswich Mass., USA) following manufacturer's instructions. Ligated vector was packaged into phages using Gigapack II Plus Packaging Extract (Agilent) following manufacturer's instructions. Recombinant phages were identified by infecting host E. coli XLR-Blue MRF′ cells with packaged product in top agar in the presence of 5-bromo-4-chloro-3-indolyl-β-D-galactopyranoside (X-gal, Sigma Aldrich) and isopropyl-β-D-thiogalactopyranoside (IPTG, Thermo Scientific, Waltham Mass., USA) as described by manufacturer. Phage containing the insert generated colorless plaques, while background plaques were blue.

Recombinant plaques were selected as follows: a clean pipette tip was inserted into the center of a colorless plaque and was rinsed in 200 ul of 0.5×SM buffer (G Biosciences, St Louis, Mo., USA). 20 ul of chloroform was added, mixed, and solution was allowed to incubate at room temperature for 2 hours. The presence of the sequence of interest was confirmed by amplifying 5 ul of the phage-containing solution using qPCR conditions described below with primers cotJC F2 and cotJC R211. Recombinant plaques were amplified in host cells and recovered in SM buffer as directed by manufacturer.

### Sputum Sample Processing and MTB Specific Capture DNA Extraction

Mycobacterial DNA was purified from sputum specimens using a sequence specific capture illustrated in FIG. 1. Two tubes of dried reagents per reaction were prepared prior to DNA extraction as described previously 11: one for sputum thinning and the second for specific capture probe (oligo) binding. Thinning buffer for study 1 was prepared such that 1% SDS (Sigma Aldrich), 30 mM Tris pH 8.0 (Thermo Scientific), and 10 mM EDTA (Ambion, Waltham Mass.) will be present after dissolution in 1 ml of sputum. For study 2, SDS was increased to 2% and EDTA was eliminated. Capture probe binding buffer for study 1 was prepared such that in 1 ml of sputum, there would be present 500 mM NaCl, 10 mM Tris pH 8.0, 1 mM EDTA, and 0.005% Tween-20 (Thermo Fisher, Waltham Mass.). For study 2, final composition of binding buffer was 300 mM NaCl, 60 mM MgCl2 (Sigma Aldrich), 10 mM Tris pH 8.0, and 0.005% Tween-20. All reagent tubes were dried on their side overnight in a 55° C. oven. After drying, the tubes were capped and stored at room temperature in aluminum moisture-barrier pouches (Ted Pella; Redding, Calif., USA), with silica gel desiccant (McMaster Carr; Elmhurst, Ill., USA) to maintain dryness and a humidity indicator card (Static Control Components; San Diego, Calif., USA) to monitor moisture.

A cocktail of biotin-labeled capture oligonucleotides was prepared in advance in which a 5 μl addition contained a total of 5.5 pmol capture probe: 2.5 pmol of capture probes targeting IS6110, 2.5 pmol targeting the senX3-regX3 region, and 0.5 pmol targeting cotJC. All capture probes were designed to target specific sequences within 100 base pairs of the amplicon. Capture probes contained a 5′ biotin moiety, included a spacer of 5 adenine residues prior to specific sequences, and were HPLC-purified. Probes were obtained from Integrated DNA Technologies (IDT, were diluted in 10 mM Tris pH 8.0, and stored at 20° C. until time of use. Proteinase K solution was prepared by combining 30 U Proteinase K (Life Technologies, Carlsbad Calif., USA) in 50 ul with 1 ul 1 M CaCl2 (Sigma Aldrich).

For study 1, Bacillus atrophaeus spore stock (104 CFU/μl; MesaLabs) was vortexed and diluted to 102 CFU/μl in 40% ethanol prior to use. For study 2, lambda phage expressing the B. atrophaeus gene cotJC was diluted to 102 copies/μl in TTG buffer [10 mM tris pH8, 0.01% tween-20, 10% glycerol (Affymetrix, Santa Clara, Calif., USA)]. Dynabeads M-270 Streptavidin paramagnetic particles (PMPs) (Life Technologies) sufficient for 20 μl per sample were washed two times in 1 ml TT buffer (10 mM tris pH8, 0.01% tween-20) and resuspended in their initial volume of TT buffer.

All clinical sputum sample processing steps prior to heat-killing of bacteria were performed in a biological safety cabinet. Positive and negative extraction controls were prepared by adding 950 μl 10 mM Tris pH8 (study 1) or 950 μl synthetic sputum (study 2) to thinning buffer tubes, with 50,000 copies of H37Rv genomic DNA added to positive extraction control tubes only. 950 μl of sputum samples were transferred into thinning buffer tubes. In cases where the specimen was not sufficient, the volume was brought to 950 μl with ultrapure water to equalize volume across the panel. Fifty-one μl of prepared Proteinase K solution and the process control were added to each sample, vortexed, and incubated for 8 minutes at 55° C. with 1500 rpm shaking in a Benchmark Multi-Therm heater-shaker (Benchmark Scientific, Edison, N.J., USA). After 3 minutes, all samples were briefly vortexed to ensure complete mixing. The heater-shaker was then set to 100° C. with 1500 rpm shaking, and samples were incubated for 10 minutes after target temperature was reached. Next, the entire volume of thinned sample was transferred to tubes containing dried binding buffer reagents. 5 μl of capture probe cocktail were added to each sample. Samples were vortexed and incubated at 60° C. for 20 minutes (study 1) or 10 minutes (study 2) with 1500 rpm shaking 11.

After the 60° C. incubation, 20 μl of washed PMPs were added to each sample, and samples were subjected to end-over-end rotation for 10 minutes at room temperature. Samples were then placed on a magnetic stand where PMPs were collected, and the supernatant was discarded. PMPs were washed in 1 ml TT and transferred to a clean tube. PMPs were collected and washed again in 1 ml TT, for a total of two washes. All remaining wash solution was carefully removed. Finally 11.75 μl of freshly-prepared TTG was used to resuspend test samples, and 20 μl was used for positive control samples. Samples were then eluted at 75° C. for 3 minutes with 1500 rpm shaking. PMPs were pelleted on a magnetic stand, and eluted DNA was transferred to a clean tube.

### PCR Amplification

Two MTB qPCR targets, IS6110 and senX3-regX3 were amplified as described in Reed, et al. 11 with modifications to oligonucleotide sequences to optimize Tm and concentration. In lieu of primer sequences, the clarified MIQE guidelines30 allow publication of the reference sequence, anchor nucleotide (defined as a nucleotide located in the probe sequence), and amplicon length for each assay: IS6110: X17348.1, 975, 174; senX3-regX3: AL123456.3, 580,840, 135; and cotJC: CP011802.1, 497,980, 85. 2.0 mM MgCl2 (Sigma Aldrich) was used. PCR mixes were prepared for a 25 μl total reaction volume (15 μl master mix and 10 μl of eluted DNA).

TTG buffer was used to prepare: a standard curve of H37Rv TMC 303 genomic DNA (105-10-1 copies/μl); 1:10 dilutions of positive extraction controls; and no template controls. 1000 copies of B. atrophaeus gBlock DNA (study 1) or lambda phage (study 2) were added as PCR positive controls for the cotJC assay. Standard curves were amplified in triplicate, and eluate from test specimens and 10× dilutions were amplified singly or in duplicate, respectively. The reactions were amplified as described previously 11, and copy number for the MTB targets relative to the standard curve was determined by using the Qiagen Rotor-Gene Q Series Software package. PCR products were frozen at −20° C. in case of discrepant results. Samples that tested negative for both the MTB assays and had a failed PRC were considered invalid. Samples that tested positive for one or both of the MTB assays with a failed PRC were considered positive.

Sanger sequencing was used to resolve discrepant samples. The Qiagen MinElute PCR Purification kit was used to purify the entire multiplexed PCR reaction, according to manufacturer's recommendations. DNA concentration was determined spectrophotometrically. PCR products were sequenced at the Northwestern University Center for Genomic Medicine Sequencing Core. DNA sequences were submitted to the NCBI BLAST website (https://blast.ncbi.nlm.nih.gov/Blast.cgi) for assessment of sequence identity and homology to known MTB species.

### LOD and Reproducibility Study

Samples for the LOD study were contrived by spiking 20, 10, or 5 cfu/ml of MTB H37Ra bacilli into a cocktail of sputum specimens prepared by vortexing 20 1 ml residual sputum specimens with approximately 7.5 g acid-washed 5 mm glass beads (Sigma Aldrich) for 5 min. 20 replicates of each concentration were evaluated following above specific capture protocol, with positive and negative controls included as described above. Samples for the reproducibility study were contrived by spiking 5000 cfu MTB H37Ra bacilli into 15 replicates of total 1 ml aliquots of three individual sputum specimens (4-6 replicates of each specimen were assayed depending on sample volume available).

### Bacillus Lysis Study

All DNA dilutions were prepared and all samples eluted in BD GeneOhm (BD GeneOhm™ Lysis Kit; Franklin Lakes, N.J., USA) sample buffer. A standard curve containing serial dilutions of cotJC gBlock® gene fragment (1e6-1e1 copies/rxn) was run as positive PCR control. No template controls and specific capture negative controls were performed, and no amplification was observed. Efficacy of the cotJC capture probes was demonstrated by extracting and amplifying 10,000 copies cotJC gBlock using the standard protocol. Prior to treatments described below, spores were centrifuged for 10 minutes at 14,000 rpm in an Eppendorf 5415c centrifuge (Hauppauge, N.Y., USA), and supernatant was removed. For each treatment, 10,000 spores were nominally present in 10 μl final volume, to be added to a 25 μl total PCR reaction.

To test for lysis of spores by heat, BD GeneOhm sample buffer was added to collected spores to a total volume of 15˜20 μl. Samples were heated for 10 minutes at 95° C. with shaking (1500 rpm). To assess lysis of spores by the GeneOhm kit, collected spores were resuspended in 50 μl BD GeneOhm sample buffer, transferred to a GeneOhm lysis tube, vortexed for 5 minutes at maximum speed (3000 rpm), and centrifuged gently to collect the liquid. The GeneOhm protocol called for samples to be incubated at 95° C. for 2 minutes with shaking (1500 rpm). To evaluate specific capture of GeneOhm-lysed spores, collected spores were resuspended and processed in GeneOhm lysis tubes as above. Entire volume was transferred to a tube containing dried thinning buffer. Water was added to 950 μl, and samples were extracted as above. To evaluate specific capture of untreated spores, collected spores were resuspended in 950 μl water, which was transferred to a tube of thinning buffer and extracted as above.

### Results

### Analytical Sensitivity of XtracTB Assay is Equivalent to Culture

The sequence specific capture probes and qPCR oligonucleotides were optimized for Tm, and the amount of NaCl was increased from 250 mM to 500 mM to enhance sequence specific capture of MTB DNA11. To estimate the limit of detection, 1 ml sputum specimens were contrived to contain 20, 10 or 5 cfu/ml of MTB H37Ra which has 17 copies of IS611016, and 20 replicates of each concentration were assayed. None of the 8 negative sputa tested were detected by either the IS6110 and senX3-regX3 assays (Table 1). All 20 of the 20 cfu/ml specimens were detected by both assays. Additionally, all 10 cfu/ml specimens were detected by the IS6110 assay, and 17 out of 20 were detected by the senX3-regX3 assay. For the 5 cfu/ml specimens, IS6110 assay detected 18 out of 20 (90%) and senX3-regX3 detected 14 out of 20 (70%). The application of two diagnostic qPCR targets allowed all 20 of the 5 cfu/ml samples to be detected, surpassing the sensitivity of either single target. Therefore, the LOD of the strain H37Ra for the combined assay was estimated to be 5 cfu/ml.

Similar Yield of MTB DNA Extracted from Buffer or Sputum

We assessed the variation of DNA yield of sequence specific extraction by testing multiple aliquots of 3 different sputum specimens and comparing the MTB DNA yield observed to spiked buffer specimens. Four to six one ml aliquots (depending upon sputum volume availability) of three residual sputum specimens were spiked with 5000 cfu/ml bacilli, and subjected to our specific capture protocol. A standard curve of MTB genomic DNA was used to calculate yields in 50% of the sample eluates as well as in a tenfold dilution of the sample eluates (FIG. 2). The yield and reproducibility of extractions as demonstrated by the coefficient of variation (CV) from each sputum specimen were very similar to that of buffer. The average yield of MTB DNA from buffer was 3.43} 0.14 log genomic copies/ml (95% CI: 3.08 to 3.77; CV=4%), and the average yield of the 3 individual sputa 3.35} 0.07 (95% CI: 3.23 to 3.47; CV=2%), 3.26} 0.04 (95% CI: 3.21 to 3.31; CV=1%) and 3.14} 0.17 (95% CI: 2.94 to 3.35; CV=5%) respectively. The results of each individual sputum specimen 15 were combined, and their average yield was 3.25} 0.13 (95% CI: 3.18 to 3.32; CV=4%). The difference in average Cq between neat eluates and tenfold dilutions (ΔCq) revealed no difference in inhibition between samples processed in buffer (ΔCq=3.87} 0.06) and samples processed in sputum (ΔCq=3.84} 0.22) with a p value of 0.71; an inhibited sample would have had a ΔCq of less than 3.

### Assessment of XtracTB Assay Using Clinical Specimens: Study One

Two 500 μl aliquots of 94 independent clinical sputum specimens acquired from the Foundation for Innovative New Diagnostics (FIND) (Geneva, Switzerland) Tuberculosis Bank were combined and 950 μl was tested in the XtracTB Assay (FIG. 3). Fifty-nine of the samples were from males and 35 were from females. Fifty-two of the specimens were collected in Peru, 22 in Vietnam and 20 in South Africa. Eighty four were HIV negative, 9 were HIV positive and there was no data for one of the patients. Twenty-nine of the specimens had lower volumes than the expected 1 ml (Supplemental Tables 1 and 2). Sample volume per specimen was normalized to 950 μl with sterile water prior to testing. Samples that tested positive for either the IS6110 or senx3-regX3 assays were considered positive. Samples that were negative for both MTB assays and were positive for the process control (PRC) were considered negative. Samples that tested negative for both MTB assays and had a failed PRC were considered invalid.

Following sample processing and data analysis, results of clinical and microbiological testing (culture and concentrated smear) for the panel were unblinded. Three of the 94 specimens (3%) were considered invalid because both MTB targets and process control failed to amplify and were excluded from further analysis (one sputum smear negative/culture positive, one scanty smear positive & one chest x-ray positive). Of the 91 valid specimens, 68 were positive by mycobacterial culture (29 smear-negative, 19 scanty smear-positive, 20 smear-positive). All 68 of these specimens were detected by the XtracTB Assay yielding 100% sensitivity (95% CI: 93.3 to 100): 68 were positive for IS6110 and 66 were positive for senX3-regX3 (Table 2; Supplemental Table 1).

Thirty-six of the culture positive specimens with valid XtracTB results (16 SSM− and 20 SSM+) had Xpert® MTB/RIF Assay result reported in FIND database. Of the 16 SSM− specimens, the Xpert test detected 12 (75.9%; 95% CI 47.4 to 91.7%) and XtracTB Assay detected 16 (100% 95% CI 75.9 to 100.0%). Of the 20 SSM+ specimens both Xpert® MTB/RIF Assay and XtracTB Assays detected 100% (95% CI 80.0 to 100.0%). Overall, the Xpert test detected 32 out of 36 (88.0%; 95% CI 73.0 to 96.4%) and the XtracTB test detected 36 out of 36 (100%; 95% CI 88.0-100.0%).

Of the 29 specimens with reduced volumes, 19 of them were from the culture negative sample pool (Supplemental Tables 1, and 2) which makes up 95% of the culture negative specimens tested. In fact, 60% of the culture negative specimens had volumes less than 50% of the 950 μl input volume. Because of the large number of specimens with low volume, this data set could not be used to establish test specificity. However, two of these culture negative specimens (75 and 89; Supplemental Table 1) were detected in both the IS6110 and senX3-regX3 assays. Eighteen of the culture negative specimens had Xpert® MTB/RIF Assay results reported in FIND database. None of the 18 was detected by the Xpert® MTB/RIF Assay (100% specificity; 95% CI 78.1 to 100.0%); this data set included the specimens 75 and 89 that were positive in the XtracTB test. Clinical notes indicated that neither of these patients had a history of TB. The chest x-ray results of one of the patients indicated pneumonia or atypical TB, but there was no other clinical or microbiological evidence supporting TB infection in these two patients. One of the samples (75; Supplemental Table 1) was a moderately strong positive, and DNA sequencing of the qPCR reaction demonstrated the presence of both the IS6110 and the senX3-regX3 amplicons. Sample 89 was a very weak positive, and only the IS6110 amplicon was detected by DNA sequencing. Taken together, these results indicate that MTB DNA was present in the samples. Three further culture-negative specimens, in which chest x-ray was positive for TB and the patients had been empirically treated for TB, were screened, and one was a very weak positive in the senX3-regX3 assay only.

### Quantification of MTB DNA in FIND Panel

The senX3-regX3 Cqs were used to estimate the number of genomic copies in the qPCR reactions from the different culture positive categories: sputum smear negative (SSM−), scanty sputum smear positive (SCA) and sputum smear positive (SSM+) (FIG. 4). The IS6110 results were not used to quantify the genomic copies detected because the number of copies of IS6110 can vary in the MTB clinical specimens 12. The specimen panel was tested on 7 different days, and on each day a standard curve of H37Rv genomic DNA was performed in triplicate. The Cq values of the standard curves were combined, and linear regression analysis was performed. The equation of the line was Y=−3.42X+35.47 (95% CI of slope −3.39 to −3.45) R2=0.99. PCR efficiency was calculated to be 96.1% demonstrating that the standard curve was highly reproducible over the 7 days of the experiment. We then calculated the log copy numbers from the Cqs of the 66 senX3-regX3 positive samples and grouped the results in categories of 10-fold dilutions (FIG. 4).

As would be expected, 95% of the SSM+ specimens were estimated to have greater than 1000 genomic copies detected per reaction with 85% of the specimens containing greater than 10,000 genomic copies. In the SCA specimen group, 79% were estimated to have between 100 and 100,000 copies per reaction but both high copy and low copy SCA samples were also detected. The SSM− population of sputum has historically been the most challenging to diagnose by molecular techniques. Of this specimen type, 52% (14/27) were estimated to have less than 100 genomic copies per reaction. However, 41% of the samples (11/27) were estimated to have greater than 1000 genomic copies of senX3-regX3 per reaction and 25% of these specimens had more than 10,000 copies per reaction. The wide range of genomic copies observed in SSM− specimens may have resulted from the inherent biological variability of sputum specimens or the variability in performance of this challenging technique. Blakemore et al. 17 also reported a substantial overlap of Cq values between smear grades indicating variability in genetic target concentration using the Xpert®MTB/RIF test and that a significant number of SSM− specimens contain relatively high numbers of MTB bacteria in their sputum.

Performance of B. atrophaeus Endospores as Process Control

Of the 94 sputum specimens tested, 22 (23%) were negative in the B. atrophaeus cotJC process control. Nineteen of these specimens were positive in at least one MTB target region with a median of 1,370 copies/sample for senX3-regX3. The remaining 3 cotJC negative specimens were classified as invalid because the two MTB targets were also negative. Furthermore, 14% of negative extraction controls performed with each run which contained buffer spiked with spores alone also failed to detect the cotJC amplicon. These data suggested a problem with detection of the process control alone, rather than assay inhibition that would ostensibly affect detection of all three targets processed together and not be a factor in the buffer controls. Although the sample processing, extraction, and PCR conditions were optimal for processing clinical TB-suspect sputa with high clinical sensitivity, this same method was not sufficient for processing B. atrophaeus spores for use as an internal extraction control. Because the results of the process control are suspect, we also calculated the clinical sensitivity including the two invalid samples which yielded clinical sensitivity for SSM−=97% (29/30; 95% CI: 80.0-99.8), SSM+=98% (39/40; 95% CI: 85.3-99.8) and combined culture positive=97% (68/70; 95% CI: 8 9.1-99.5).

### Bacillus Spores are Inefficiently Lysed by Heat Step

The inconsistent results of the process control assay could have been the result of poor performance of the qPCR assay, inconsistent capture of the target, or inefficient lysis of the spores. Standard curve of 10-1,000,000 copies of cotJC gBlock® demonstrates that assay is sensitive and linear (FIGS. 5A and B), and capture of 10,000 copies of cotJC gBlock® yielded a mean of 9385} 411 copies (94%) (FIG. 5A) demonstrating that both the assay and the specific capture based extraction performed as anticipated.

The lysis step in the sequence specific capture protocol involves heating specimens in the heater/shaker set at 100° C. for 10 minutes. To determine if this was sufficient to lyse B. atrophaeus endospores, this strategy was compared to the BD GeneOhm Lysis Kit which has the reported lysis efficiency of Bacillus endospores of 98.8% (package insert http://www.bd.com/ds/productCenter/441243.asp). The GeneOhm kit yielded an average Cq of 22.3} 0.9 compared to an average Cq of 30.5} 1.2 for heat alone (FIG. 5C). Two-tailed Student t test indicated that the means were significantly different (p=1.2e-7.) These lysed samples were also subjected to DNA extraction via specific capture yielding an average Cq 23.5} 0.5 for the GeneOhm lysed samples and 30.1} 1.5 for the standard sample preparation treatment, and the Cq means were significantly different (p=1.4e-6) The Cq differences between the GeneOhm kit lysed spores and the heat lysed spores indicates that the GeneOhm kit lyses ˜200 times more spores than heating for 10 minutes at 100° C. Therefore, we concluded that the 95° C. lysis step used in the sequence specific capture extraction protocol was not sufficient to efficiently release genomic DNA from Bacillus endospores and that a revamped process control was necessary to monitor the lysis of pathogens during the nucleic acid purification procedure. The DNA that we were able to detect in our previous studies was most likely free DNA from bacterial cell lysate becoming associated with the outside of spores 18.

### Revamped Process Control Introduced

Using traditional lambda phage cloning techniques, we generated phage particles that express the cotJC amplicon from B. atrophaeus (used above) and therefore we were able to continue to use the same multiplexed MTB qPCR assay. One thousand lambda phage particles acting as a control for cell lysis, nucleic acid extraction and qPCR amplification 19 were added to sputum containing a range of MTB DNA from 0 to 50,000 genomic copies/ml. The average Cq of the cotJC assay was 27.4} 0.3 (range 26.9-28.2) across the range of MTB concentrations used indicating consistent performance.

### Assessment of Revamped XtracTB Assay Using Clinical Specimens: Study Two

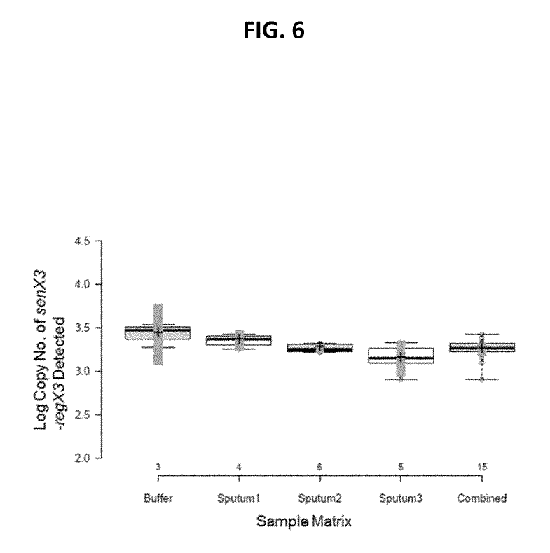

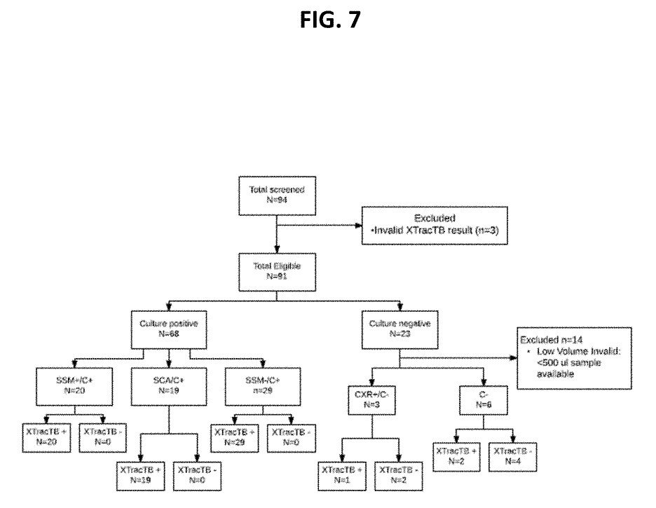

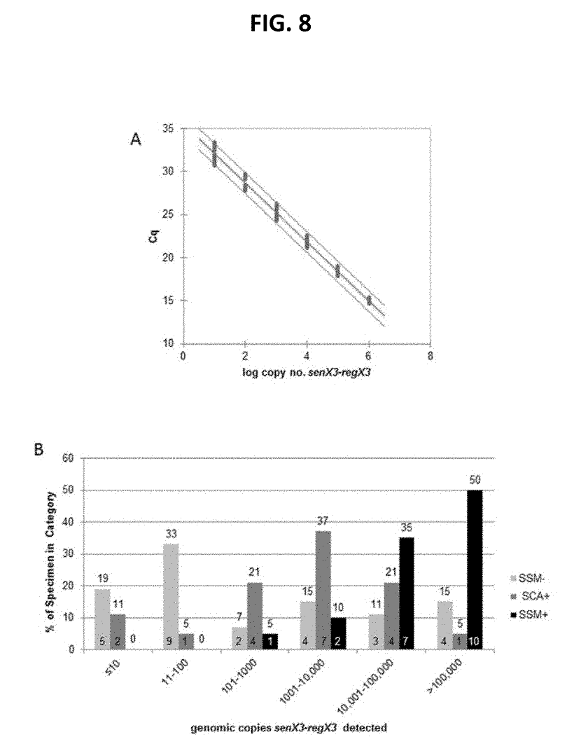

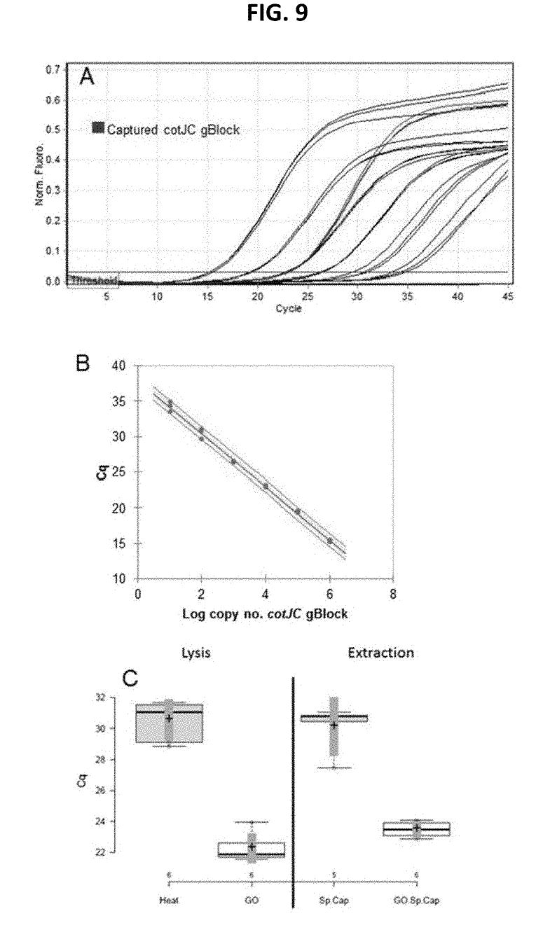

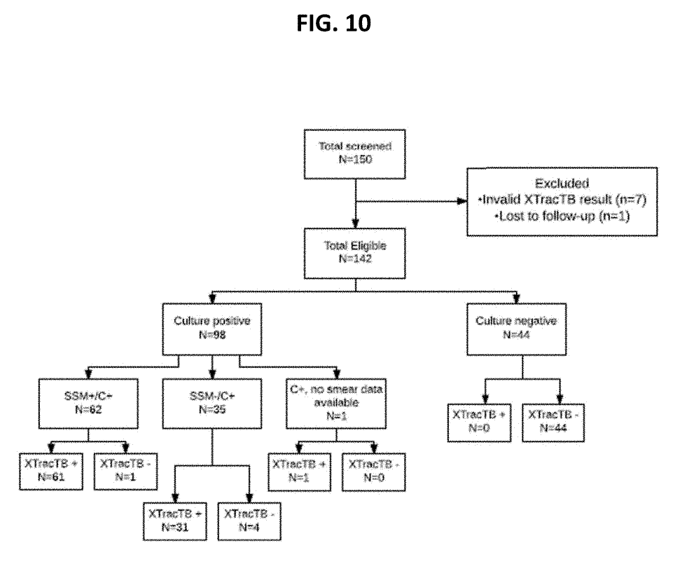

To validate this new assay design and to establish test specificity, a second panel of 150 MTB specimens from FIND was tested (FIG. 6) using a slightly modified extraction protocol to shorten the DNA hybridization time from 20 minutes to 10 minutes. Seven of the samples were invalid in the XtracTB test due to negative TB and PRC results (5%) and one sample was eliminated by FIND due to loss to follow-up leaving 142 samples with valid test results. Eighty-six of the samples were from males and 56 were from females. One hundred and twenty four of the specimens were collected in Peru, 14 in Vietnam, and 4 in South Africa. One hundred and twelve were HIV negative, 28 were HIV positive and there was no HIV data for 2 of the patients. Only 6 of the specimens had associated Xpert® MTB/RIF Assay results. Twenty of the specimens had lower volumes than the expected 1 ml (Supplemental Table 3). Sample volume per specimen was normalized to 950 μl with sterile water prior to testing. Following sample processing and data analysis, results of clinical and microbiological testing for the panel were unblinded.

Of the 142 valid specimens, 98 were positive by mycobacterial culture (35 SSM−, 62 SSM+, and one with unknown smear status), and 44 were culture negative. Sixty-one of the 62 SSM+ specimens were detected with XtracTB assay yielding a clinical sensitivity of 98.4% (95% CI: 90.1-99.9), and 31 out of 35 of the SSM− were detected (88.6%; 95% CI: 72.3-96.3) (Table 3). The overall sensitivity of the test was 94.9% (95% CI: 87.9-98.1). None of the 44 culture negative specimens were detected by XtracTB for a clinical specificity of 100% (95% CI: 90.0-100).

### Example 9

### Optimized Primers for senX3-regX3 Assay

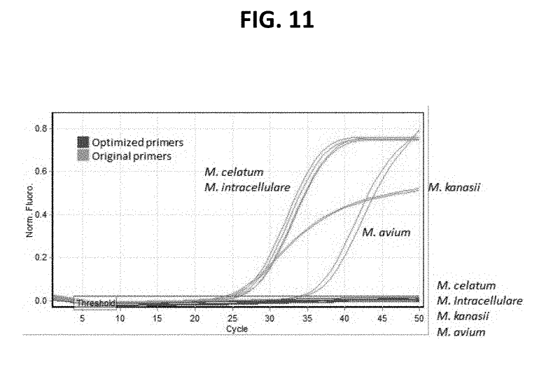

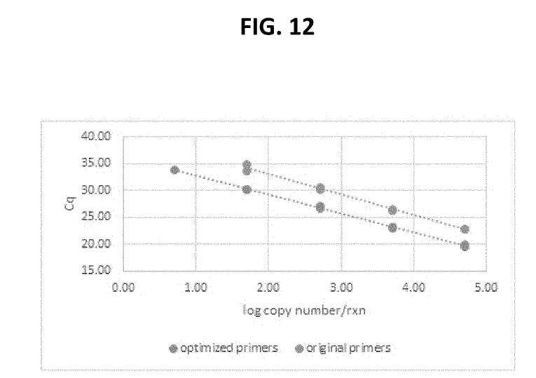

The senX3-regX3 primers used in some experiments conducted during development of embodiments herein exhibited a low level of cross reactivity with non Mycobacterium tuberculosis (NTM) species, M. avium, M. celatum, M. intracellulare, and M. kansasii. Sequence alignments of senX3-regX3 sequences from these NTMs were evaluated and several sets of primers were designed that would maximize mismatches without obliging us to replace the probe. F11 and R4.1 were selected because no amplification of NTMs was observed (FIG. 11) and an approximately 3 Cq improvement in amplification was observed with these optimized primers compared to the original primer set (FIG. 12).

F11:
(SEQ ID NO: 41)
5′-TGACCAGTGTGTTGATTGTG-3′
R4.1:
(SEQ ID NO: 42)
5′-AGAGCTGCCGGACCATC-3′
Probe 10:
(SEQ ID NO: 43)
5′-/5HEX/AGG ACG AGG/ZEN/AGT CGC TGG C/3IABkFQ/-3′

All publications and patents provided herein are incorporated by reference in their entireties. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

1.-33. (canceled)

34. A method for preparing a biological sample for analysis, comprising: mixing the biological sample with thinning reagents comprising a detergent and a proteinase to produce a lysis mixture.

35. (canceled)

36. The method of claim 35, wherein the detergent comprises one or more anionic detergents selected from the group consisting of sodium dodecyl sulfate (SDS), N-lauroylsarcosine sodium salt, and sodium deoxycholate.

37. The method of claim 35, wherein the detergent comprises one or more neutral detergents selected from the group consisting of CHAPS and CHAPSO.

38. The method of claim 35, wherein the proteinase is proteinase K (PK).

39. The method of claim 35, wherein the biological sample is selected from the group consisting of sputum, whole blood, mucus, nasal fluid, semen, saliva, amniotic fluid, and bronchial fluid.

40. (canceled)

41. The method of claim 34, wherein the biological sample is undiluted prior to the mixing.

42. The method of claim 41, wherein the thinning reagents are liquid reagents and have a volume that is less than 25% of the volume of the biological sample.

43. (canceled)

44. The method of claim 43, wherein the dried reagents are adhered to a surface of a vessel to which the biological sample is added.

45. The method of claim 34, wherein cells within the biological sample have not been lysed prior to the mixing.

46. The method of claim 34, wherein the biological sample has not been subjected to sonication or chaotropic agents.

47. The method of claim 34, further comprising heating the lysis mixture.

48.-49. (canceled)

50. The method of claim 49, wherein a bacterial pathogen present in the biological sample is not pathogenic in the lysis mixture following heating.

51. A method comprising:
(a) preparing a biological sample for analysis by the method of one of claims 34-51; and
(b) extracting nucleic acids from the lysis mixture.

52. The method of claim 51, wherein extracting nucleic acids from the lysis mixture comprises:
combining the lysis mixture with hybridization buffer and at least one capture oligonucleotide to generate a capture solution, wherein the at least one oligonucleotide is specific for a target sequence and is linked to a capture moiety; and
(ii) contacting the capture solution with a capture agent, wherein the capture agent binds to the capture moiety
wherein a capture complex is formed comprising (A) a nucleic acid comprising the target sequence, (B) the capture oligonucleotide, and (C) the capture agent.

53. The method of claim 51, wherein extracting nucleic acids from the lysis mixture comprises combining the lysis mixture with hybridization buffer and at least one capture oligonucleotide to generate a capture solution, wherein the at least one oligonucleotide is specific for a target sequence and is linked to a capture moiety, wherein the oligonucleotide is displayed a solid surface, and wherein a capture complex is formed comprising (A) a nucleic acid comprising the target sequence and (B) the capture oligonucleotide displayed on the solid surface.

54.-56. (canceled)

57. The method of claim 52, wherein the capture agent and the capture moiety form a stable non-covalent interaction upon contact.

58. The method of claim 57, wherein the capture agent is bound to a solid surface.

59. The method of claim 53 or 58, wherein the solid surface is a particle.

60. (canceled)

61. The method of claim 52, further comprising:
(iii) separating the capture complex from the capture solution.

62.-63. (canceled)

64. The method of claim 61, further comprising:
(iv) eluting the nucleic acid comprising the target sequence from capture complex into an elution buffer.

65.-74. (canceled)
